SEQUENCE LISTING (41 sequences)

SEQ ID NO: 1 - protein, 233 aa, Porcine circovirus
MTYPRRRYRRRRHRPRSHLGQILRRRPWLVHPRHRYRWRRKNGIFNTRLSRTFGYTVKATTVRTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWMRLQTSRNVDHVGLGTAFENSIYDQDYNIRVTMYVQFREFNLKDPPLKP

SEQ ID NO: 2 - protein, 233 aa, Porcine circovirus
MTYPRRRYRRRRHRPRSHLGQILRRRPWLVHPRHRYRWRRKNGIFNTRLSRTFGYTIKRTTVRTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWLRLQTAGNVDHVGLGTAFENSIYDQEYNIRVTMYVQFREFNLKDPPLNP

SEQ ID NO: 3 - protein, 18 aa, Porcine circovirus
YTVKATTVRTPSWAVDMM

SEQ ID NO: 4 - protein, 18 aa, Porcine circovirus
WPCSPITQGDRGVGSTAV

SEQ ID NO: 5 - protein, 18 aa, Porcine circovirus
ILDDNFVTKATALTYDPY

SEQ ID NO: 6 - protein, 18 aa, Porcine circovirus
VLDSTIDYFQPNNKRNQL

SEQ ID NO: 7 - protein, 27 aa, Porcine circovirus
YTVKATTVRTPSWAVDMMRFNIDDFVP

SEQ ID NO: 8 - protein, 45 aa, Porcine circovirus
RIRKVKVEFWPCSPITQGDRGVGSTAVILDDNFVTKATALTYDPY

SEQ ID NO: 9 - protein, 27 aa, Porcine circovirus
VLDSTIDYFQPNNKRNQLWMRLQTSRN

SEQ ID NO: 10 - protein, 8 aa, Porcine circovirus
LKDPPLKP

SEQ ID NO: 11 - protein, 12 aa, swine influenza virus
EALMEWLKTRPI

SEQ ID NO: 12 - protein, 24 aa, Porcine circovirus
KATTVRTPSWAVDMMRFNIDDFVP

SEQ ID NO: 13 - protein, 8 aa, Porcine circovirus
TNKISIPF

SEQ ID NO: 14 - protein, 6 aa, Porcine circovirus
KATTVR

SEQ ID NO: 15 - protein, 18 aa, Porcine circovirus
RIRKVKVEFWPCSPITQG

SEQ ID NO: 16 - protein, 18 aa, Porcine circovirus
DRGVGSTAVILDDNFVTK

SEQ ID NO: 17 - protein, 9 aa, Porcine circovirus
WPCSPITQG

SEQ ID NO: 18 - protein, 10 aa, Porcine circovirus
KATALTYDPY

SEQ ID NO: 19 - protein, 9 aa, Porcine circovirus
RIRKVKVEF

SEQ ID NO: 20 - protein, 15 aa, Porcine circovirus
VLDSTIDYFQPNNKR

SEQ ID NO: 21 - protein, 15 aa, Porcine circovirus
DNFVTKATALTYDPY

SEQ ID NO: 22 - protein, 12 aa, Porcine circovirus
STIDYFQPNNKR

SEQ ID NO: 23 - protein, 18 aa, Artificial
  Synthetic peptide - modified decoy epitope 3
ILDDNFVNKSTALTYDPY

SEQ ID NO: 24 - protein, 15 aa, Artificial
  Synthetic peptide - modified decoy epitope 4
VLDSTIDYFNPNNSR

SEQ ID NO: 25 - protein, 18 aa, Porcine circovirus
ILDDNFVTKANALTYDPY

SEQ ID NO: 26 - protein, 15 aa, Porcine circovirus
VLDRTIDYFQPNNKR

SEQ ID NO: 27 - protein, 17 aa, Neospora caninum
QSSEKRDGEQVNKGKPP

SEQ ID NO: 28 - DNA, 21 bp, Artificial
  qPCR primer
aagtgggaggtttgcctttgt

SEQ ID NO: 29 - DNA, 20 bp, Artificial
  qPCR primer
atggcccaatcctcggagaa

SEQ ID NO: 30 - DNA, 19 bp, Artificial
  qPCR probe
tacctgttccccgtcgcgt

SEQ ID NO: 31 - DNA, 25 bp, Artificial
  qPCR primer
ggcctacatggtctacatttccagt

SEQ ID NO: 32 - DNA, 23 bp, Artificial
  qPCR primer
ggtactttaccccgaaacctgtc

SEQ ID NO: 33 - DNA, 29 bp, Artificial
  qPCR probe
tgggttggaagtaatcgattgtcctatca

SEQ ID NO: 34 - protein, 8 aa, Porcine circovirus
ILDDNFVT

SEQ ID NO: 35 - protein, 7 aa, Porcine circovirus
FTPKPVL

SEQ ID NO: 36 - protein, 10 aa, Porcine circovirus
FQPNNKRNQL

SEQ ID NO: 37 - protein, 9 aa, Porcine circovirus
KATTVRTPS

SEQ ID NO: 38 - protein, 9 aa, Porcine circovirus
CSPITQDRG

SEQ ID NO: 39 - protein, 6 aa, Porcine circovirus
DNFVTK

SEQ ID NO: 40 - protein, 4 aa, Porcine circovirus
TYDP

SEQ ID NO: 41 - protein, 234 aa, Porcine circovirus
MTYPRRRFRRRRHRPRSHLGQILRRRPWLVHPRHRYRWRRKNGIFNTRLSRTIGYTVKKTTVRTPSWNVDMMRFNINDFLPPGGGSNPLTVPFEYYRIRKVKVEFWPCSPITQGDRGVGSTAVILDDNFVTKANALTYDPYVNYSSRHTITQPFSYHSRYFTPKPVLDRTIDYFQPNNKRNQLWLRLQTTGNVDHVGLGTAFENSIYDQDYNIRITMYVQFREFNLKDPPLNPK

(19) United States
(12) Patent Application Publication
(10) Pub. No.: US 2022/0265808 A1
(43) Pub. Date: Aug. 25, 2022

(54) A PORCINE CIRCOVIRUS TYPE 2 (PCV2) VACCINE

(71) Applicants: NDSU Research Foundation, Fargo, ND (US); Biological Mimetics, Frederick, MD (US)

(72) Inventors: Sheela RAMAMOORTHY, Fargo, ND (US); Peter NARA, Frederick, MD (US)

(21) Appl. No.: 17/628,403

(22) PCT Filed: Jul. 27, 2020
(86) PCT No.: PCT/US2020/043770

§ 371 Date: Jan. 19, 2022

Related U.S. Application Data
(60) Provisional application No. 62/879,016, filed on Jul. 26, 2019.

(51) Int. Cl. A61K 39/12; C07K 14/01; A61P 31/14

(52) U.S. Cl. CPC A61K 39/12; C07K 14/01; A61P 31/14; A61K 2039/523

(57) ABSTRACT

A PCV2 vaccine and a method of vaccinating against PCV2 are provided herein. The PCV2 vaccine includes a PCV2 infectious clone with a re-engineered PCV2 capsid in the backbone thereof, wherein the re-engineered PCV2 capsid includes a modified immunogenic region. The method of vaccinating against PCV2 includes administering the PCV2 vaccine including a PCV2 infectious clone with a re-engineered PCV2 capsid in the backbone thereof to a subject in need thereof.

### RELATED APPLICATIONS

This application claims the benefit of U.S. Provisional Application Ser. No. 62/879,016, filed Jul. 26, 2019, the entire disclosure of which is incorporated herein by this reference.

### GOVERNMENT INTEREST

This invention was made with government support under grant nos. 2014-31100-06038, 2015-67016-23318, and NI18HMFPXXXXG008 awarded by the United States Department of Agriculture/National Institute of Food and Agriculture (USDA/NIFA). The government has certain rights in the invention.

### SEQUENCE LISTING

The instant application contains a Sequence Listing which has been submitted in ASCII format via EFS-Web and is hereby incorporated by reference in its entirety. The ASCII copy of the Sequence Listing, which was created on Jul. 27, 2020, is named NO137-576WO.txt and is 13.8 kilobytes in size.

### TECHNICAL FIELD

The presently-disclosed subject matter generally relates to a vaccine for porcine circovirus type 2 (PCV2). In particular, certain embodiments of the presently-disclosed subject matter relate to altered PCV2 vaccines and methods for developing altered vaccines.

### BACKGROUND

Porcine circoviruses (PCVs) consist of the non-pathogenic porcine circovirus strain 1 (PCV1) and the pathogenic porcine circovirus strain 2 (PCV2) types. Porcine circovirus type 2 is a small, single-stranded DNA virus, with a circular genome and relatively high plasticity. It is an economically important swine virus which causes post-weaning multi-systemic wasting syndrome (PMWS) and lymphadenopathy in weanling piglets, along with a range clinical signs such as jaundice, nephropathy, reproductive and respiratory disorders collectively known as porcine circovirus associated diseases (PCVAD).

The approximately 1700 bp PCV2 genome encodes just two major proteins; the replicase (ORF1) and capsid (ORF2) proteins. The capsid protein is considered to be both necessary and sufficient for the prevention of PCV2, as subunit vaccination with the capsid protein alone is effective at preventing clinical signs. Hence, while the cell mediated immune response to PCV2 is not well studied, neutralizing antibody responses targeted to the capsid protein are considered to be critical for protection against PCV2. Strong binding Ab responses to PCV2 can be detected as early as 7 days post infection in naturally or experimentally infected pigs. However, neutralizing responses, whose appearance correlates with a reduction in viremia, are not detected until later in infection.

Infections characterized by delayed virus neutralizing Ab responses commonly present decoy epitopes, which are characterized by sequence variability, hydrophilicity, structural flexibility and proximity to conserved, functionally important regions such as receptor binding sites. Decoy epitopes are usually immunodominant and divert the Ab responses away from neutralizing epitopes. Immuno-dominance is the phenomenon by which the immune system preferentially mounts responses to selected antigens, or epitopes within antigens, and is an effective immuno-subversion mechanism for pathogens and a well-established confounding factor in the development of effective vaccines. While several studies on epitope mapping of the PCV2 capsid protein have identified four major immunodominant regions containing over-lapping linear and conformational epitopes, fewer studies have characterized the functionality of the identified epitopes. Of those regions which have been characterized, conformational neutralizing epitopes have been mapped to residues 47-58, 165-200, and 230-233. However, only one decoy epitope spanning residues 169-180 has been identified so far.

Despite the remaining need for a more complete picture of possible immuno-subversive strategies, several commercial vaccines against PCV2 are available and commonly deployed in pork production units. Most of the commercial vaccines continue to target the first discovered PCV2 subtype, PCV2a (SEQ ID NO: 1), either as whole inactivated virus, inactivated chimeric PCV1-2a virus preparations, or subunits of the capsid protein. Although existing vaccines are effective at preventing clinical signs of PCV2 and in reducing economic damage due to the virus, they do not prevent transmission or shedding of PCV2. As such, vaccinated animals continue to be viremic, transmitting the virus both horizontally and vertically. Additionally, since the introduction of commercial vaccines, the initially predominating PCV2a subtype was replaced by a 2^{nd} subtype, PCV2b (SEQ ID NO: 2), and more recently by PCV2d (SEQ ID NO: 41). Therefore, it is possible that selection pressure induced by commercial vaccines could be driving viral evolution in the field.

Together, the delayed production of neutralizing Ab responses, coupled with the periodical emergence of new PCV2 subtypes following vaccination, suggests that antibody based immunodominance plays an important role in PCV2 pathogenesis and vaccine mediated protection. Thus, there remains a need for both a more complete picture of possible immune-subversive strategies as well as vaccines which enable differentiation of vaccinated and infected animals (DIVA) to facilitate possible eradication of PCV2 in the long term, and to ensure vaccine compliance during routine production.

Further, live attenuated vaccines against PCV2 may be more effective than current inactivated or subunit vaccines. However, attenuated PCV2 vaccines are not used in the field due to the need for a high safety margin to prevent reversion to virulence.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a PCV2 vaccine including a PCV2 infectious clone with a re-engineered PCV2 capsid in the backbone thereof, wherein the re-engineered PCV2 capsid includes a modified immunogenic region. In some embodiments, the PCV2 infectious clone is selected from the group consisting of PCV2a (SEQ ID NO: 1), PCV2b (SEQ ID NO: 2), and PCV2d (SEQ ID NO: 41). In some embodiments, the modified immunogenic region includes at least one modification as compared to a region selected from the group consisting of wild type region 1, wild type region 2, wild type region 3, wild type region 4, and combinations thereof.

In some embodiments, the modified immunogenic region includes at least one modification to a decoy epitope sequence contained therein. In some embodiments, the decoy epitope sequence is selected from the group consisting of SEQ ID NO: 3, SEQ ID NO: 4, SEQ ID NO: 5, SEQ ID NO: 6, SEQ ID NO: 17, SEQ ID NO: 18, SEQ ID NO: 20, SEQ ID NO: 25, SEQ ID NO: 26, and combinations thereof. In some embodiments, the decoy epitope sequence is selected from the group consisting of SEQ ID NO: 3, SEQ ID NO: 17, SEQ ID NO: 18, and combinations thereof. In some embodiments, the decoy epitope sequence is selected from the group consisting of SEQ ID NO: 4, SEQ ID NO: 5, SEQ ID NO: 20, and combinations thereof. In some embodiments, the modified immunogenic region includes at least one modification to each of SEQ ID NO: 5 and SEQ ID NO: 20. In some embodiments, the modified immunogenic region includes at least two modifications to each of SEQ ID NO: 5 and SEQ ID NO: 20. In some embodiments, the decoy epitope sequence is selected from the group consisting of SEQ ID NO: 25, SEQ ID NO: 26, and a combination thereof. In some embodiments, the modified immunogenic region includes a modified decoy epitope sequence selected from the group consisting of SEQ ID NO: 23, SEQ ID NO: 24, and a combination thereof.

In some embodiments, the re-engineered PCV2 capsid further comprises at least one modified serine or modified leucine codon, wherein the modified serine codon include at least one mutation selected from the group consisting of UCA to UAA, UCA to UGA, and UCG to UAG, and wherein the modified leucine codon include at least one mutation selected from the group consisting of UUA to UAA, UUA to UGA, and UUG to UAG. In some embodiments, each serine and leucine codon is modified. In some embodiments, the mutation converts the at least one modified serine or modified leucine to a stop codon.

In some embodiments, the vaccine further comprises a marker for differentiating infected and vaccinated animals (DIVA). In some embodiments, the DIVA marker includes a peptide that is foreign to swine. In some embodiments, the DIVA marker includes SEQ ID NO: 27.

Also provided herein, in some embodiments, is a method of vaccinating against PCV2, the method including administering the vaccine according to one or more embodiments disclosed herein to a subject in need thereof. In some embodiments, after administration of the PCV2 infectious clone with the re-engineered PCV2 capsid in the backbone thereof refocus the immune response in the subject towards more protective regions on the capsid protein. In some embodiments, the method further comprises determining whether the subject is infected using the DIVA marker and removing infected subject from the herd.

Further features and advantages of the presently-disclosed subject matter will become evident to those of ordinary skill in the art after a study of the description, figures, and non-limiting examples in this document.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1 shows images illustrating the location of putative decoy epitopes. Regions with potential decoy activity identified in Table 1 mapped to the crystal structure of the PCV2 capsid protein [PDB-3R0R]. Cyan—C terminal, Magenta—N terminal, residues 55-63—Yellow, residues 106-113—Blue, residues 133-141—Brown, residues 169-180—Red. Surface diagram generated using EzMol.

FIG. 2 shows an image identifying immunodominant regions of the PCV2 capsid protein. Sequence alignment of the capsid proteins of PCV2a strain 40895 and PCV2b strain 16845. Solid boxes—Four major immunodominant regions, Dark bars—putative decoy epitopes identified in this study, T—decoy epitope identified by Trible et. al.

FIGS. 3A-B show graphs illustrating reactivity of post-vaccination sera to peptides. Reactivity of pooled serum collected at 35 days post-vaccination from pigs (N=8) vaccinated with either (A) an inactivated or (B) subunit commercial PCV2 vaccine, with a peptide library spanning the 233 amino acid long PCV2 capsid protein by ELISA. Y axis—mean signa/negative (S/N) ratio, X axis—peptide number. Values above the black bar at a value of 1 on the Y axis are considered positive.

FIGS. 4A-D show images illustrating viral replication of the PCV2b virus encoding mutations to target suicidal replication of the vaccine virus (MLV-I). The mutated PCV2b virus culture was rescued by transfection and used to infect PK-15 monolayers for 3 passages. Viral replication was assessed by staining the cell sheet with a PCV2 specific monoclonal antibody. (A) Mutated PCV2b with DIVA marker of infected cells, showing the nuclear green fluorescence. (B) Shows the negative control stained with PCV2b specific antibody. (C) Mutated PCV2b with DIVA marker infected cell showing the nuclear green fluorescence stained with anti-Neospora caninum antibody. (D) Negative control, stained with anti-Neospora caninum antibody.

FIGS. 5A-D show images illustrating viral replication of the PCV2b virus encoding mutations to selected decoy epitopes (MLV II) in PK-15 monolayers. The mutated PCV2b virus culture was rescued by transfection and used to infect PK-15 monolayers for 3 passages. Viral replication was assessed by staining the cell sheet with a PCV2 specific monoclonal antibody. (A) Mutated PCV2b with DIVA marker of infected cells, showing the nuclear green fluorescence. (B) Shows the negative control stained with PCV2b specific antibody. (C) Mutated PCV2b with DIVA marker infected cell showing the nuclear green fluorescence stained with anti-Neospora caninum antibody. (D) Negative control, stained with anti-Neospora caninum antibody.

FIG. 6 shows an image illustrating multiple sequence alignment of the PCV2 capsid protein: Selected amino acid sequences of the PCV2 capsid protein representing the major circulating subtypes PCV2a, b and d, generated using the Jal View 2.4 software. Boxes represent epitope A and B. Conserved residues are indicated by dots.

FIG. 7 shows an image illustrating a map of the rPCV2-Vac construct. Diagrammatic representation of the PCV2b infectious clone showing the PCV2b genome, major open reading frames, location of Epitope A and B and the insertion site of the DIVA tag as an independent transcriptional unit in the 5′ end of the capsid gene (ORF2).

FIG. 8 shows a graph illustrating PCV2a, PCV2b, and PCV2d virus neutralization in MLV-I vaccinated pigs, MLV-II vaccinated pigs, pigs vaccinated with commercial vaccine (Merial), and unvaccinated pigs at 28 days post vaccination.

FIGS. 9A-B show antibody responses to the mutated epitopes. Loss of immunodominant effects due to mutation of epitopes A and B as qualitatively assessed by surface plasmon resonance. 20 μM of purified IgG was tested for all experimental antisera. (A) Responses to a peptide encoding the wildtype epitope A. (B) Responses to a peptide encoding wildtype epitope B. Slashed line—anti-serum to the wildtype virus, dotted line—anti-serum to the commercial vaccine, solid line—anti-serum to the rPCV2-Vac, curved dashes—anti-serum from the unvaccinated group.

FIGS. 10A-B show an image and graph illustrating verification of the DIVA marker peptide and measurement of antibody responses to the SRS2 DIVA peptide. (A) Western blot of the purified DIVA marker peptide. Left lane—Molecular weight marker, Right lane—Purified protein detected by a monoclonal anti-HIS tag antibody. (B) Antibody responses to the SRS2 DIVA peptidein MLV-I vaccinated, MLV-II vaccinated, commercial control (Merial), and unvaccinated control groups.

FIG. 11 shows graphs illustrating challenge virus replication 9 and 21 days post challenge with a virulent, heterologous PCV2d strain in MLV-I vaccinated pigs, MLV-II vaccinated pigs, pigs vaccinated with commercial vaccine (Merial), and unvaccinated pigs.

FIGS. 12A-G shows graphs illustrating tissue lesion scores in various tissues. (A) Assessment of the pathology resulting from challenge viral replication is represented as the sum of the scores for lymph nodes tissue. (B) Assessment of the pathology resulting from challenge viral replication is represented as the sum of the scores for spleen tissue. (C) Assessment of the pathology resulting from challenge viral replication is represented as the sum of the scores for tonsils tissue. (D) Assessment of the pathology resulting from challenge viral replication is represented as the sum of the scores for ileum tissue. (E) Assessment of the pathology resulting from challenge viral replication is represented as the sum of the scores for lung tissue. (F) Consolidated score for all tissues. In A-F Gross lung lesions were scored from 0-100% to represent the % area of affected lung. Microscopic lesions were scored with a scale of 1-4; where 1=single follicle or focus staining 2=rare to scattered staining, 3=moderate staining 4=strong widespread staining. X axis—groups, Y axis—scores, dots—values for the individual pigs, horizontal bar with the large circle—group mean, bars—95% confidence interval of the means, *-significantly different from the PBS group, @*-significantly different from the commercial vaccine group, (p<0.05) by a Mann Whitney U test. (G) Assessment of the pathology in the lungs, lymph nodes, tonsils, and ileum of MLV-I vaccinated pigs, MLV-II vaccinated pigs, pigs vaccinated with commercial vaccine (Merial), and unvaccinated pigs that were challenged with a virulent, heterologous PCV2d strain.

FIG. 13 shows graphs illustrating anti-PCV2 IgG responses. Mean signal to positive (S/P) ratios of sera collected on days 0, 14 and 28 post vaccination (DPV) and on days 9- and 21-days post-challenge (DPC), as measured by a PCV2 specific commercial ELISA. X axis—time points of serum collection, Y axis—Signal to positive (S/P) ratio, Dotted line—Commercial vaccine, Solid line—rPCV2-Vac, hashed line—Unvaccinated. Error bars indicate the standard deviation, * significantly different from the unvaccinated control, p≤0.05, Students T test.

### DEFINITIONS

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials are described below.

Following long-standing patent law convention, the terms “a,” “an,” and “the” refer to “one or more” when used in this application, including the claims. Thus, for example, reference to “a cell” includes a plurality of cells, and so forth.

The terms “comprising,” “including,” and “having” are intended to be inclusive and mean that there may be additional elements other than the listed elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, percentage, or the like is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

All combinations of method or process steps as used herein can be performed in any order, unless otherwise specified or clearly implied to the contrary by the context in which the referenced combination is made.

### DETAILED DESCRIPTION

The details of one or more embodiments of the presently-disclosed subject matter are set forth in this document. However, modifications to embodiments described in this document, and other embodiments, will be evident to those of ordinary skill in the art after a study of the information provided in this document. As such, it should be understood that the description of specific embodiments is not intended to limit the disclosure to cover all modifications, equivalents and alternatives falling within the spirit and scope of the disclosure as defined by the appended claims. Instead, the information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes articles and methods for vaccinating against porcine circovirus type 2 (PCV2). In some embodiments, the articles include a PCV2 vaccine including a reengineered PCV2 capsid in the backbone thereof. In some embodiments, the reengineered PCV2 capsid includes modifications (e.g., mutations) to linear decoy epitopes that are conserved or substantially conserved between PCV2 subtypes. As used herein, the phrase “substantially conserved between PCV2 subtypes” means that the corresponding linear decoy epitope(s) include no more than 2 mismatched amino acids between subtypes. For example, the decoy epitopes spanning amino acids 124-141 (SEQ ID NO: 5) and 166-180 (SEQ ID NO: 20) of PCV2a are conserved in PCV2b (i.e., they are identical), and are substantially conserved in PCV2d, with each containing a single amino acid mismatch as shown in SEQ ID NO: 25 and SEQ ID NO: 26, respectively.

As will be appreciated by those skilled in the art, since the linear decoy epitopes being modified are conserved or substantially conserved between subtypes, any PCV2 subtype may serve as the backbone for the PCV2 vaccine. For example, in one embodiment, the PCV2 vaccine includes a PCV2a infectious clone with a reengineered PCV2 capsid in the backbone thereof. In another embodiment, the PCV2 vaccine includes a PCV2b infectious clone with a reengineered PCV2 capsid in the backbone thereof. In a further embodiment, the PCV2 vaccine includes a PCV2d infectious clone with a reengineered PCV2 capsid in the backbone thereof.

Any suitable conserved or substantially conserved linear decoy epitope in the PCV2 subtype may be modified to form the reengineered PCV2 capsid backbone. In some embodiments, the vaccine includes at least one modification to the PCV2a (SEQ ID NO: 1), PCV2b (SEQ ID NO: 2), PCV2d (SEQ ID NO: 41), or other PCV2 capsid protein. In some embodiments, the vaccine includes at least two modifications to the PCV2a (SEQ ID NO: 1), PCV2b (SEQ ID NO: 2), PCV2d (SEQ ID NO: 41), or other PCV2 capsid protein. In some embodiments, the modifications are to an immunogenic region of the PCV2 capsid. For example, in one embodiment, the vaccine includes at least one modification to region 1, 2, 3, and/or 4 (TABLE 1). In another embodiment, the at least one modification is to a major immunogenic region having a sequence according to SEQ ID NO: 7, 8, 9, and/or 10. In a further embodiment, the at least one modification is to an immunodominant decoy epitope having a sequence according to SEQ ID NO: 3, 4, 5, and/or 6. In one embodiment, the vaccine includes at least two modifications to region 1, 2, 3, and/or 4 (TABLE 1). In another embodiment, the at least two modifications are to a major immunogenic region having a sequence according to SEQ ID NO: 7, 8, 9, and/or 10. In a further embodiment, the at least two modifications are to an immunodominant decoy epitope having a sequence according to SEQ ID NO: 3, 4, 5, and/or 6. As will be appreciated by those skilled in the art, the immunodominant decoy epitope sequences according to SEQ ID NOS: 3-6 are within the major immunogenic regions according to SEQ ID NOS; 7-9, and thus any modification to an immunodominant decoy epitope will also be considered a modification to the overlapping immunogenic region.

TABLE 1
Immunogenic regions of the PCV2 capsid

| | | | | |
|---|---|---|---|---|
| | SEQ | | | Time point |
| | ID | Sequence and | Peptide | of detection |
| Region | NO | location | No | (DPI) |

Regions with decoy activity

| | | | |
|---|---|---|---|
| 1 | 3 | 55 YTVKATTVRTPS | 19-21 |
| | | WAVDMM 72 | |
| 2 | 4 | 106 WPCSPITQGDR | 36-38 |
| | | GVGSTAV 123 | |
| 2 | 5 | 124 ILDDNFVTKAT | 42-44 |
| | | ALTYDPY 141 | |
| 3 | 6 | 166 VLDST1DYFQP | 56-57 |
| | | NNKRNQL 183 | |

Major Immunogenic regions

| | | | | |
|---|---|---|---|---|
| 1 | 7 | 55 YTVKATTVRTPSW | 20-24 | 7, 14, 21, 28 |
| | | AVDMMRFNIDDFVP 81 | | |
| 2 | 8 | 97 RIRKVKVEFWPCS | 33-44 | 7, 14, 21, 28 |
| | | PITQGDRGVGSTAVIL | | |
| | | DDNFVTKATALTYDP | | |
| | | Y 141 | | |
| 3 | 9 | 166 VLDSTIDYFQPN | 56-61 | 7, 14, 21, 28 |
| | | NKRNQLWMRLQTSR | | |
| | | N 192 | | |
| 4 | 10 | 226 LKDPPLKP 233 | 73-75 | 21, 28 |

In some embodiments, the modifications are to decoy epitope sequences such as, but not limited to, SEQ ID NO: 3, SEQ ID NO: 4, SEQ ID NO: 5, SEQ ID NO: 6, SEQ ID NO: 17, SEQ ID NO: 18, SEQ ID NO: 20, SEQ ID NO: 25, and/or SEQ ID NO: 26. For example, in one embodiment, the reengineered PCV2 capsid includes at least one modification to YTVKATTVRTPSWAVDMM (SEQ ID NO: 3), WPCSPITQG (SEQ ID NO: 17), and/or KATALTYDPY (SEQ ID NO: 18). Additionally or alternatively, in one embodiment, the reengineered PCV2 capsid includes at least one modification to SEQ ID NO: 4, SEQ ID NO: 5, and/or SEQ ID NO: 20. In another embodiment, the reengineered PCV2 capsid includes two modification to each of SEQ ID NO: 5 and SEQ ID NO: 20. In one embodiment, the reengineered PCV2 capsid includes at least one modification to SEQ ID NO: 25 and/or SEQ ID NO: 26. In some embodiments, the reengineered PCV2 capsid includes SEQ ID NO: 23 or SEQ ID NO: 24. In some embodiments, the reengineered PCV2 capsid includes SEQ ID NO: 23 and SEQ ID NO: 24.

In some embodiments, the PCV2 capsid is also mutated such that the vaccine virus undergoes suicidal replication in the host. This eliminates the possibility of vaccine-induced disease or recombination with field strains to produce new variants. Serine and leucine amino acids are encoded by 6 redundant codons each. Of these 6 codons, two codons for each amino acid (UUA, UUG for Leucine and UCA, UCG for Serine) require just one single mutation to be converted to a stop codon. To increase the chances of a stop codon occurring during viral replication in the pigs, all the serine and leucine amino acids of the capsid protein of the vaccine virus were redesigned as in Table 2.

TABLE 2
Redesigning the serine and leucine codons

| | WT codons | | |
|---|---|---|---|
| | Original codons | Redesigned to | Stop |
| | in gene | 1-to-stop Codon | Codon |
| Serine | UCU, UCC, | UCA | UAA & |
| | AGU, AGC, | UCG | UGA |
| | | | UAG |
| Leucine | CUA, CUG, | UUA, | UAA & |
| | CUU, CUC | UUG | UGA |
| | | | UAG |

Additionally or alternatively, in some embodiments, the PCV2a infectious clone with the reengineered PCV2 capsid also includes a marker for differentiating infected and vaccinated animals (DIVA). Suitable DIVA markers include, but are not limited to, peptides which are “foreign” to swine. For example, in one embodiment, the marker includes a highly immunogenic, 18 amino acid long segment from the surface antigen-1 related sequence 2 (SRS2) protein (AAD04844.1) of N. caninum. In another embodiment, the marker includes Amino acids 324 QSSEKRDGEQVNKGKPP 348 (SEQ ID NO: 27) of the SRS2 protein. In some embodiments, the marker has an antigenicity index score sufficient to ensure that it will not cross react serologically with other swine related proteins. In some embodiments, the marker is inserted into the 5′ end of the capsid gene of the PCV2 vaccine disclosed herein.

Also provided herein, in some embodiments, is a method of vaccinating swine against PCV2. In some embodiments, the method includes administering one or more of the articles disclosed herein to a swine. In one embodiment, after administration the modifications to the one or more immunodominant decoy epitopes refocus the immune response in the swine towards more protective regions on the capsid protein, as compared to PCV2 capsids without the immunodominant decoy epitope modifications. In some embodiments, administration of the articles disclosed herein provides a lower total IgG Ab response against the capsid protein as compared to existing commercial vaccines (e.g., vaccines without one or more modified immunodominant decoy epitopes), while providing a clear anamnestic response. In some embodiments, the administration of the articles disclosed herein may be used to vaccinate against any PCV2 strain, such as, but not limited to, PCV2a, PCV2b, and/or PCV2d. Additionally or alternatively, in some embodiments, the method includes determining whether the swine is infected using the DIVA marker, and removing infected swine from the herd.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1

Porcine circovirus type 2 (PCV2) is an economically important swine virus which causes post-weaning multisystemic wasting syndrome (PMWS) in weanling piglets. Commercial vaccines against PCV2 are highly effective. Yet, a recurring emergence of new subtypes in vaccinated herds necessitates a better understanding of protective immunity. As such, this Example is directed to identifying previously unrecognized decoy epitopes in the PCV2 capsid protein and demonstrating that early antibody responses map to potential decoy epitopes and vice versa. Since virus neutralizing Ab responses are not detected until later in PCV2 infection, the premise that the earliest detected immunodominant Ab responses in PCV2 infected animals would correspond to potential decoy epitopes is also discussed herein. Further discussed herein is the identification of immune-subversive regions of the capsid protein which dominate the early Ab response in PCV2 infection.

Using a peptide library spanning the PCV2a capsid protein (SEQ ID NO: 1) and weekly sera collections from PCV2a infected animals, three major immunodominant regions mapping to the early responses were identified. Regions with potential decoy activity were further narrowed down using peptide blocking fluorescent focus inhibition assays to residues 55 YTVKATTVRTPSWAVDMM 72 (SEQ ID NO: 3), 106 WPCSPITQGDRGVGSTAV 123 (SEQ ID NO: 4), and 124 ILDDNFVTKATALTYDPY 141 (SEQ ID NO: 5). Post-vaccination responses also largely recognized the three identified regions, which appeared to dominate the antibody responses to PCV2 in both infection and vaccination.

Material and Methods

Peptides, antibodies, and viruses: A peptide library spanning the entire 233 amino acids of the capsid protein (ORF2) of PCV2a strain 40895 (GenBank Accession AF264042) was commercially synthesized (Mimotopes, Victoria, Australia) as overlapping 12mer biotinylated peptides with a 3 aa overlap (total 75 peptides). Serum was collected weekly from 3-week-old, PCV2 negative piglets which were experimentally infected with PCV2a strain 40895 as previously described. Sera from 12 pigs collected on days post infection (DPI) 0, 7, 14, 21 and 28 were pooled for the assessment of binding antibody responses to the peptides. Similarly, sera collected at 35 days post-vaccination (DPV) from 8 pigs each, which were vaccinated with either a commercial inactivated or subunit PCV2 vaccine were used to assess post-vaccination Ab responses to the pep-set. All sera used in the study were previously tested with PCV2 capsid protein specific ELISAs. To prepare pure cultures of the virus for the virus neutralization assays, an infectious clone of PCV2a strain 40895 was used to rescue recombinant virus cultures as described previously.

Detection of antibody responses to the peptide library: An indirect ELISA format was used for the detection and differentiation of the early and mature PCV2 Ab responses to biotinylated peptide library spanning the PCV2 capsid protein. The same protocol was used to test post-vaccination Ab responses to the individual peptides. To coat the ELISA plates (Maxisorp, Nalge Nunc, Rochester, N.Y.), 100 μl of a 10 μg/ml solution of streptavidin in sterile distilled water was added to the wells and allowed to dry overnight. After washing 5 times with phosphate buffered saline with Tween20 (PBST) containing 2% BSA, the plates were the incubated with 100 μl of a 10 μg/ml solution of each biotinylated peptide at 37° C. for 1 hr. Plates were then blocked with 2% BSA, 2% skimmed milk powder and 2% normal goat serum in PBST for 2 hrs at 37° C. Test samples were prepared by pooling equal volumes of sera from twelve PCV2 infected pigs collected at DPI 0, 7, 14, 21 and 28 each or DPV 35 sera from 8 pigs each administered a commercial inactivated or subunit vaccine. Each pool was then was diluted to 1:50 in PBST containing 2% BSA and added in 100 μl volumes to the peptide coated plates and incubated for 1 hr at 37° C. After washing 5 times with PBST, anti-swine IgG HRPO conjugate (KPL, Gaithersburg, Md.) at a 1:5000 dilution in blocking buffer was added plates incubated at 37° C. for 1 hr. Detection was achieved using the tetramethylbenzidine (TMB) substrate (KPL, Gaithersburg, Md.) and incubation in the dark for 15 mins at room temperature. Finally, 1M HCl was added to stop the reaction. Optical density (OD) readings were obtained at 450 nm using a microplate reader (BioTek Instruments, Winooski, Vt.). All samples were assessed in duplicate. The mean signal to negative [S/N] ratio for each peptide was calculated as the OD value for each peptide divided by the corresponding value of the day 0 sample. Values above an S/N ratio of 1 were considered positive (Table 1).

Virus neutralization assay: A conventional virus neutralization (V/N) assay format was used to obtain the V/N titers for the pooled samples as described before, with some modifications. Each of the pooled sera, prepared as described above, was serially diluted two-fold from 1:2 to 1:1024 dilutions in PBS, in sterile U bottom plates. The PCV2a strain 40895 virus culture was diluted to 10^{3.5}TCID₅₀/ml, and equal volumes added to the diluted sera. The U bottom plates were incubated for 1 hr at 37° C. The mixture was then layered on pre-formed PK-15 cells at 60% confluence in 96 well tissue culture plates. Virus replication was visualized after 36 hrs by staining with a PCV2-specific monoclonal antibody as previously described. The virus neutralization titer was determined as the log₂ serum dilution at which 80% or higher reduction in the number of fluorescent foci was noted, when compared to the virus only control.

Virus neutralizing activity of peptides: To localize virus neutralizing activity within the immunodominant regions identified by the pep-scan ELISA, a peptide-blocked fluorescent focus neutralization [FFN] assay was performed essentially as described before, with some modification. Blocking of virus neutralizing Abs by a peptide was expected to increase virus replication and hence, the number of fluorescent foci detected, and vice versa. To block the activity of Abs specific to the peptides, a pool of 5-6 peptides [20-23 aa total] spanning the length of each identified immunogenic region was first tested.

To prepare the pool, equal volumes of a 1 μg/ml solution of each peptide was mixed well. Each pool (10 μl) was incubated for 60 mins at 37° C. with 50 μl heat inactivated, pooled DPI 28 PCV2a anti-serum or pooled DPV35 serum at a 1:4 dilution in PBS. A non-specific swine-influenza virus-specific peptide [EALMEWLKTRPI] (SEQ ID NO: 11) and DPI 0 serum were used as controls. The PCV2a culture was adjusted to 100-150 fluorescent focus units/well and 50 μl was mixed with the peptide blocked antisera, followed by incubation at 37° C. for 60 mins. The serum/peptide/virus mixtures were incubated for 36 hrs on preformed PK-15 monolayers at 60% confluence, in 8 well chamber slides.

Virus replication was visualized by a PCV2-specific immunofluorescence assay, as previously described. The number of fluorescent foci in each well was counted in a blinded fashion by two individuals, in two independent experiments, with 3 replicates for each peptide pool (total 12 values). Activity was assessed as the mean percentage change in the number of fluorescent foci in the sample blocked with peptides, when compared to the unblocked DPI 28 PCV2 antiserum. To further narrow down the residues involved, smaller pools of 2-3 peptides spanning the regions identified to have potential decoy activity in the first screen were tested next. Each peptide pool was tested in 4 replicates and 2 independent experiments (total 8 values). All other procedures were similar to the initial screen (Table 1).

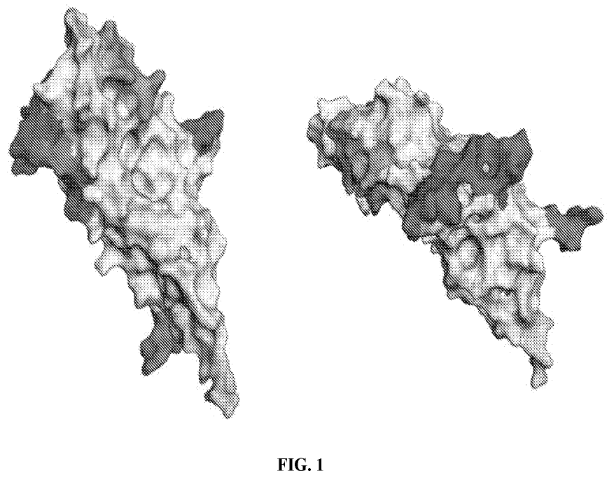

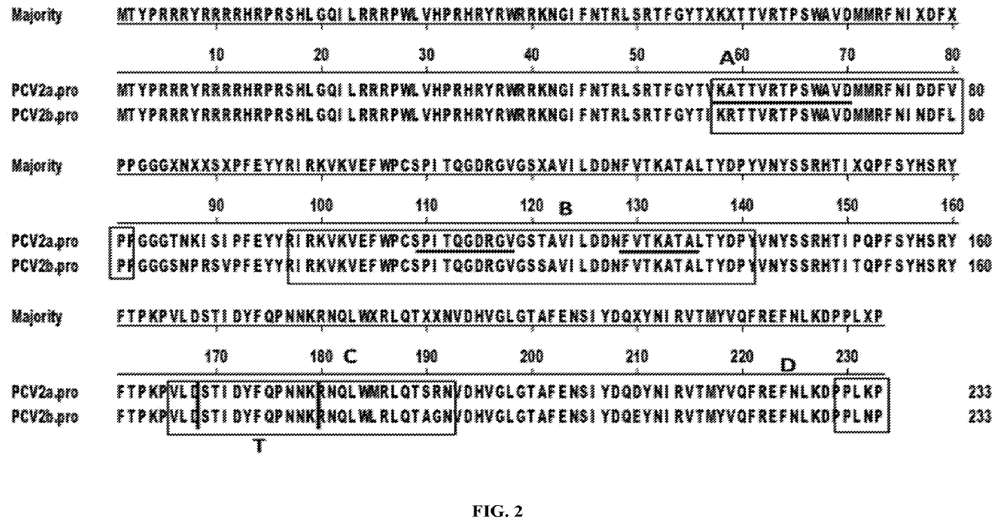

Pairwise statistical differences at p<0.05 between the blocked and unblocked serum for each peptide pool was assessed by the Mann Whitney U test. To determine location and surface exposure of the aa identified as having potential decoy activity, the residues were visualized on the crystal structure of the monomeric unit of the PCV2 capsid protein (PDB ID 3R0R) using the EzMol molecular visualization tool (FIG. 1) and on an alignment of representative PCV2a and 2b capsid protein sequences (FIG. 2).

Results and Discussion

Early antibody responses map to three major immunogenic regions. To test the premise that the early Ab responses in PCV2 infected animals would be directed towards non-protective regions of the PCV2 capsid protein, the differential antibody responses between early and late infection were characterized using the pep scan ELISA and post-infection sera collected at weekly intervals. In agreement with our previous findings, PCV2-specific Ab responses were detected as early as DPI 7, although the magnitude of the responses was low. Early Ab responses mapped to peptides 20-24 (58 KATTVRTPSWAVDMMRFNIDDFVP 81) (SEQ ID NO: 12), 33-46 (97 RIRKVKVEFWPCSPITQGDRGVGSTAVILDDNFVTKATALTYDPY 141) (SEQ ID NO: 8) and 56-61 (166 SGSGVLDSTIDYFQPN NKRNQLWMRLQTSRN 192) (SEQ ID NO: 9) (FIG. 2, Table 1). The strongest binding Ab responses were detected against peptides 19-20 and 56-61, which contained a decoy epitope previously identified by Trible et. al. (FIGS. 1-2). Virus neutralizing (V/N) Ab titers were not detected DPI 0 or 7.

The Ab responses to the three regions persisted and increased in strength at DPI 14. In addition, weak responses to peptides 74-75 containing the N terminal amino acids (226 LKDPPLKP 233) (SEQ ID NO: 10) were observed, suggesting the residues could participate in the formation of a neutralizing epitope. The V/N titer at DPI 14 was 1:8. Between DPI 21 and 28, responses to all four regions increased in magnitude (FIG. 2, Table 1). Virus neutralization increased to 1:32 and 1:64 respectively. The four major antigenic regions detected in this study corresponded to the immunodominant regions previously identified by others (FIG. 2). The signature motif sequence [86 TNKISIPF 93 (SEQ ID NO: 13), peptides 28-29] which can genetically distinguish the PCV2a, b and d subtypes was not antigenic. Unlike Guo et. al who detected an immuno-dominant epitope in the N terminal nuclear localization signal, the first 40 aa were not immunogenic in this study. Thus, it was expected that the three major immunodominant regions which reacted with the DPI 7 serum would contain putative decoy epitopes.

Mapping of virus neutralizing activity: To determine which peptides would be able to block Abs with virus neutralizing activity, pools of 5-6 peptides spanning the identified immunodominant regions were reacted with the DPI 28 serum pool. The extent of blocking was visualized as an increase or decrease in viral replication in a fluorescent focus neutralization assay. Overall, potential decoy activity appeared to localize to residues 58-160 (peptides 19-43), while protective activity was detected between residues 160-233 (peptides 51-75). Possible decoy activity were detected for peptide pools 19-24, 33-38 and 39-43 with values for peptides 33-39 being statistically significant. When 2-3 peptides were used instead of 5-6 peptides in the 2^{nd} screen to narrow down the regions responsible for the identified activity, peptides 19-21 [55 YTVKATTVRTPSWAVDMM 72] (SEQ ID NO: 3) showed potential decoy activity, while peptides 22-24 did not. The aa sequence 59 KATTVR 64 (SEQ ID NO: 14) was previously identified as immunodominant in studies where linear or conformational epitopes were mapped, with residues 59 and 60 being critical for subtype or strain specific reactivity. These residues were previously found to map to Abs with neutralizing activity. However, interestingly, when residues 59 and 60 were mutated neutralizing activity was significantly improved in vitro, indicating that the identified epitope could actually be a decoy epitope, as identified in this study.

In the second immunodominant region spanned by peptides 33-44 (Table 1) peptides 33-35 (97 RIRKVKVEFWPCSPITQG 114) (SEQ ID NO: 15) and 39-41 (115 DRGVGSTAVILDDNFVTK 132) (SEQ ID NO: 16) either blocked neutralizing activity or had no activity in the 2^{nd} screen using fewer peptides. Hence, it could be deduced that decoy activity was localized to 106 WPCSPITQG 114 (SEQ ID NO: 17) and 132 KATALTYDPY 141 (SEQ ID NO: 18), while neutralizing activity could be attributed to 97 RIRKVKVEF 105 (SEQ ID NO: 19). Indeed, a putative receptor binding site function has been proposed for residues RIRKVK. The location of neutralizing epitopes, adjacent to decoy epitopes resulting in steric interreference with Ab binding to the neutralizing epitope as a mechanism of immune evasion has been described before.

The third immuno-dominant region, 166 VLDSTIDYFQPNNKRNQLWMRLQTSRN 192 (SEQ ID NO: 9) spanning peptides 56-61 (Table 1), contained the decoy epitope (166 VLDSTIDYFQPNNKR 180) (SEQ ID NO: 20) identified by Trible et. al. This region showed very strong responses to the DPI 7 serum, which persisted for the duration of the study on the pep scan analysis. However, no significant decoy activity was detected in the first screen. When the peptides containing the core epitope (peptides 55 and 56) and key residues (173 YFQ 175, 179 K) alone were tested separately, the activity in the FFN assay was non-neutralizing. However, the values were not statistically significant. These findings are in agreement with Lekcharoensuk et. al., who found that residues 165-200 could interact with residues 58-63 to form conformational neutralizing epitopes. Hence, without wishing to be bound by theory, it is believed that overlapping linear and conformational epitopes are present in this location.

The 4^{th} immunodominant region which was recognized later in infection spanned peptides 70-75 and contained a previously identified neutralizing epitope involving the last 3 aa “231 LKP 233.” The three N terminal residues, “231 LKP 233,” often vary between newly emerging subtypes, with several PCV2b strains having the sequence LNP, and the more recently emerged PCV2d having a single N terminal amino acid elongation to LKPK.

Mapping of the residues to the crystal structure using PDB structure ID 3R0R and the EzMol molecular visualization tool showed that of the three putative decoy epitopes identified in this study, residues 55 YTVKATTVRTPSWAVDMM 72 (SEQ ID NO: 3) [FIG. 1 Yellow, FIG. 2—solid lines], and 106 WPCSPITQGDRGVGSTAV 123 (SEQ ID NO: 4) [FIG. 1—Blue, FIG. 2—solid lines] were surface exposed and adjacent to the five-fold axis. Residues 127 DNFVTKATALTYDPY 141 (SEQ ID NO: 21) [FIG. 1—brown, FIG. 2—solid lines] also mapped to a linear epitope which was partially surface exposed. Residues KATTVRTPS (SEQ ID NO: 37), CSPITQDRG (SEQ ID NO: 38), DNFVTK (SEQ ID NO: 39), and TYDP (SEQ ID NO: 40) were located in the loop regions connecting the β sheets. Confirming previous findings by Trible et al., the decoy epitope 169 STIDYFQPNNKR 180 (SEQ ID NO: 22) [FIG. 1—Red, FIG. 2—T] mapped largely to the interior of the capsid in the assembled virus like particle. In a previous study where we had computationally predicted PCV2 epitopes contributing to subtype specific immunity, three epitopes each were predicted within the 1^{st} and 2nd regions and one epitope in the 3^{rd} region, while the 4^{th} region was not predicted as immunogenic by the programs used. Hence, computational tools for B cell epitope prediction, while requiring experimental validation for accuracy, can be useful in guiding epitope analysis. Of the potential decoy epitopes identified in this study WPCSPITQG (SEQ ID NO: 17) was conserved between subtypes PCV2a, b and d while the others were variable.

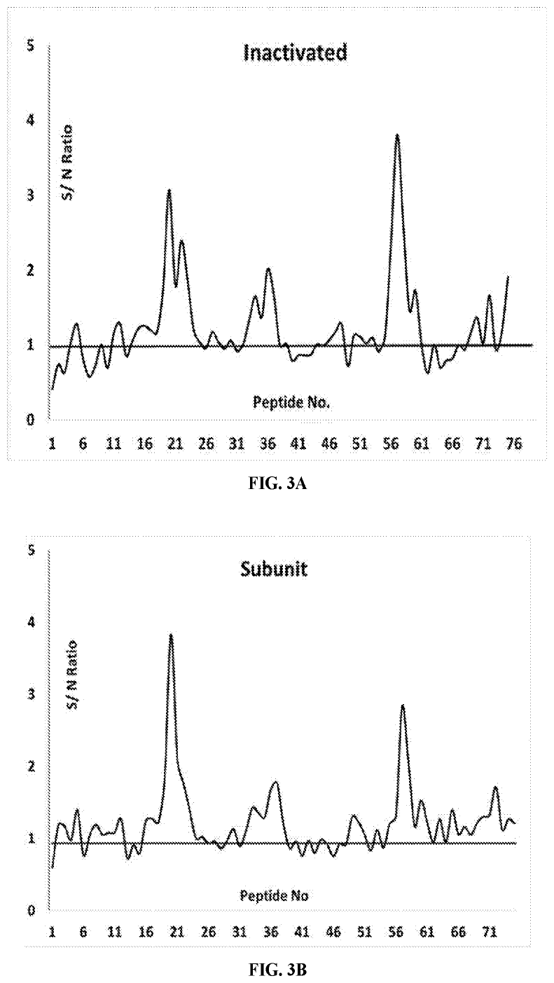

Antibody responses in vaccinated pigs: When post-vaccination serum from pigs administered either an inactivated or subunit vaccine was tested on the pep scan to assess differential responses between infection and vaccination, the trends were similar in infected and vaccinated animals, with the higher magnitude responses being directed towards immunodominant regions 1 [residues 55 YTVKATTVRTPSWAVDMM 72] (SEQ ID NO: 3) and 3 [residues 166 VLDSTIDYFQPNNKRNQL 183] (SEQ ID NO: 6). Responses to region 2 were low with detectable responses to residues 106 WPCSPITQGDRGVGSTAV 123 (SEQ ID NO: 4) but not 124 ILDDNFVTKATALTYDPY 141 (SEQ ID NO: 5) (FIGS. 3A-B, Table 1).

Trible et al., found that vaccination with the monomeric form of the capsid protein induced high levels of Abs to the decoy epitope, 169 STIDYFQPNNKR 180 (SEQ ID NO: 22), while vaccination with the fully assembled VLP did not. The level of Abs to this epitope was found to correlate inversely with neutralizing Abs in vaccinated animals. However, significant differences in the pattern of responses between the inactivated and subunit vaccines were not found in this Example. Instead, the findings in this Example are in agreement with Worsfold et. al., who also found strong Ab responses to this epitope in vaccinated pigs, with the strength of the response increasing with the age of the pigs. While regions with neutralizing activity were not characterized in this study, only low magnitude responses to the C-terminal aa with known neutralizing activity were detected in vaccinated animals (FIGS. 3A-B), suggesting that a majority of Abs produced by vaccination may not contribute to protective immunity. However, since PCV2 vaccines are very effective at preventing clinical manifestation of the disease, the level of protective Abs induced could be sufficient to achieve clinical protection. Alternately, cell mediated immunity against PCV2, which is under-studied, may play a major role in vaccine induced protection.

Thus, three new PCV2 capsid protein sequences, YTVKATTVRTPSWAVDMM (SEQ ID NO: 3), WPCSPITQG (SEQ ID NO: 17), and KATALTYDPY (SEQ ID NO: 18), with possible immuno-subversive activity were identified in this Example. The data described supports the belief that the earliest detectable Ab responses in PCV2 infected pigs will likely localize to decoy epitopes. It also supports the broader premise that the approaches used in this Example can be applied to other pathogens with a delayed virus neutralizing Ab response to characterize Ab responses at the linear epitope level. Hence, the data and approaches described in this Example contribute to further understanding PCV2 Ab mediated immunity.

### Example 2

Despite the availability of commercial vaccines which can effectively prevent clinical signs, porcine circovirus type 2 (PCV2) continues to remain an economically important swine virus, as strain drift followed by displacement of new subtypes occurs periodically. Commercial vaccines against PCV2 were introduced in the U.S in 2006. They solely target the PCV2a subtype and are effective in preventing clinical signs. However, the recent viral evolution and emergence of new PCV2 strains suggest that the existing vaccines require updating or improvement in efficacy. While antibody responses to the PCV2 capsid protein are considered to be both necessary and sufficient for protection, as discussed in Example 1 above, a significant portion of the early antibody responses are non-functional, thus serving as a host immune-evasion mechanism. More specifically, the present inventors had previously determined that the early antibody responses to the PCV2 capsid protein in infected pigs map to immunodominant but non-protective, linear B cell epitopes of the PCV2 capsid protein.

With that in mind, the primary objective of this Example was to determine if the threshold of protection against PCV2 can be improved by further rationalization of current vaccine design. This included mapping the putative protective and non-protective regions of the PCV2 capsid protein and then reengineering the PCV2 capsid in the backbone of a PCV2b infectious clone, such that the immune response is refocused towards more protective regions on the capsid protein. Using sequential anti-sera from infected pigs and a panel of overlapping peptides spanning the PCV2 capsid protein, 3 new linear, immunodominant but non-protective regions of the PCV2 capsid protein were identified and the presence of a previously identified immuno-dominant decoy epitope was confirmed. It was also found that a majority of the Abs produced by vaccination mapped to the non-protective, immunodominant epitopes identified. Based upon these findings, the present inventors tested the hypothesis that abrogation of the immunodominance patterns induced by two of the previously identified, non-protective epitopes would raise the threshold of protection attained PCV2 by vaccination. More specifically, to further improve PCV2 vaccine efficacy, two of the previously identified immunodominant epitopes were mutated in the backbone of a PCV2b infectious clone to rationally restructure the immunogenic viral capsid protein. The rescued virus was used to immunize 3-week-old weanling piglets, followed by challenge with a virulent heterologous PCV2d strain.

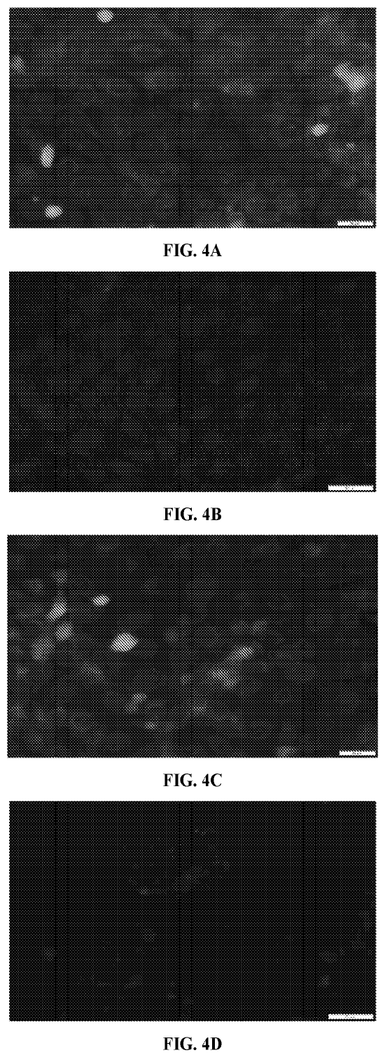

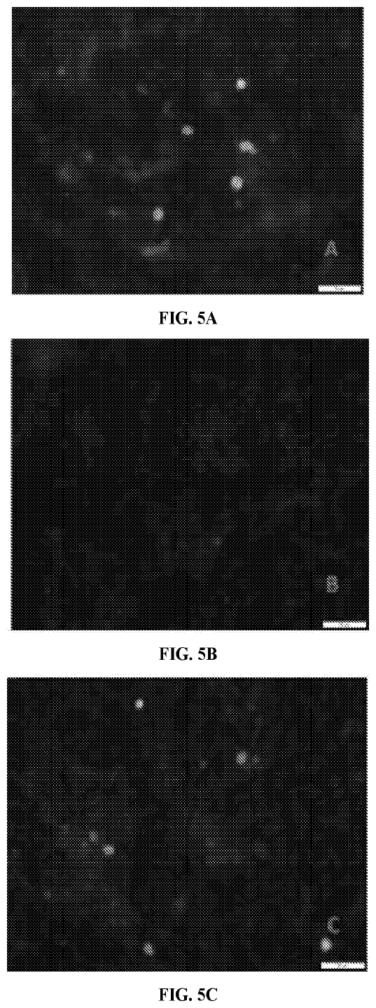

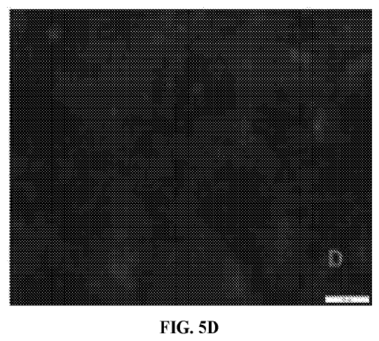

Additionally, in veterinary medicine, the successful eradication of an infectious disease requires a vaccine that is both effective and has the capability of differentiating infected and vaccinated animals (DIVA). DIVA vaccines are usually accompanied by an immuno-assay which can help to differentiate infected and vaccinated animals. Infected animals which are removed from the herd eventually lead to a disease free population. However, none of the current PCV2 vaccines have DIVA capabilities, nor is a PCV2 DIVA immuno-assay available. As such, a secondary objective of this Example was to develop a marker vaccine against PCV2 by introducing an immunogenic foreign peptide in the vaccine construct, to enable detection (Absof antibodies) against the marker to distinguish between vaccinated and infected pigs (i.e., to serve as a DIVA marker). This construct was designated as modified live vaccine I (MLV-I) (FIGS. 4A-D). To further enhance vaccine safety, mutations were introduced in the capsid, such that the vaccine virus would undergo suicidal replication in the host, eliminating the possibility of vaccine-induced disease or recombination with field strains to produce new variants. This construct was designated MLV-II (FIGS. 5A-D).

Vaccination of pigs with the restructured PCV2b vaccine (rPCV2-Vac) encoding a DIVA marker, which is also referred to herein as MLV-II, and challenge with the currently predominating heterologous PCV2d strain resulted in improved heterosubtypic virus neutralization responses, protection against tissue pathology, lack of viremia due to the challenge virus, improved weight gain, and Ab responses specific to the DIVA tag. More specifically, a loss of immunodominant antibody responses to the targeted epitopes and an overall reduction in the magnitude of the antibody responses was detected. The loss of immunodominance to the targeted epitopes correlated with a broadening of the virus neutralization responses and absence of tissue pathology in the lymphoid organs. Challenge viral replication was detected in only 1/7 pigs at day 21 post-challenge. Thus, as hypothesized, rational redesign of the PCV2 capsid antigen resulted an alteration of the immunodominance hierarchy and improved PCV2 vaccine performance. Accordingly, the strategy described in this Example provides insights into the mechanisms of vaccine mediated protection against PCV2 and has long term implications for improving the control and prevention of PCV2.

Material and Methods

Cells and viruses: The PCV1 free porcine kidney cell line, PK-15N (005-TDV, National Veterinary Services Laboratory, Ames, Iowa, USA), was used to culture all PCV2 strains. An infectious clone of PCV2b strain 41513 (GenBank accession number KR816332) was used as the backbone for the vaccine. An infectious clone of a heterologous PCV2d strain (GenBank accession number JX535296.1) was used to prepare the challenge virus. For virus neutralization assays, infectious clones of PCV2a (AF264042.1), PCV2b (EU340258.1), and PCV2d (JX535296.1) were used to generate virus stocks by transfection as described below.

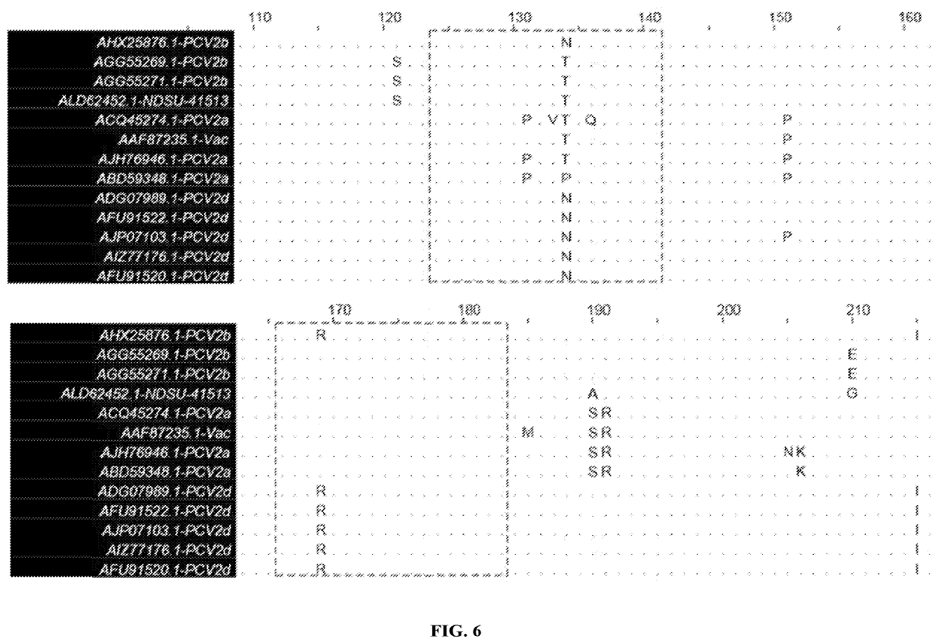

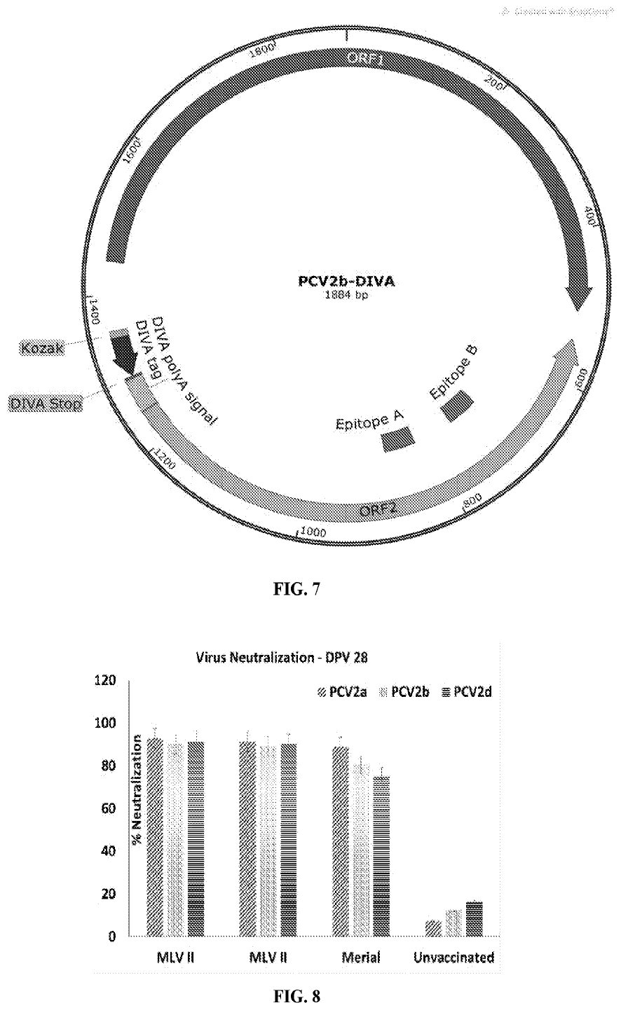

Cloning of the vaccine construct: Using the infectious clone of PCV2b 41513 as the backbone, two previously identified linear immuno-dominant, but non-protective epitopes in the immunogenic PCV2 capsid protein were mutated. The capsid gene segment encoding the desired mutations was commercially synthesized, and cloned into the backbone of PCV2b 41513 by restriction digestion. To minimize the risk of producing a lethal mutation, selected amino acids in the linear decoy epitopes were replaced with other amino acids with a low penalty score on a point accepted mutation (PAM) matrix as follows; Epitope A—124 ILDDNFVTKATALTYDPY 141 (SEQ ID NO: 5) was modified to 124 ILDDNFVNKSTALTYDPY 141 (SEQ ID NO: 23), and Epitope B—166 VLDSTIDYFQPNNKR 180 (SEQ ID NO: 20) was modified to 166 VLDSTIDYFNPNNSR 180 (SEQ ID NO: 24) (Table 3, FIGS. 6-7). All mutations were validated by sequencing (Eurofin Genomic, USA). The vaccine construct is henceforth referred to as the re-structured PCV2 vaccine (rPCV2-Vac) throughout the Examples.

TABLE 3
Amino acid sequences of Epitope A and B

| Subtype | Epitope A | Epitope B |
|---|---|---|
| PCV2a | 124 ILDDNFVT | 166 VLDSTIDY |
| (AF264042.1) | KATALTYDPY 141 | FQPNNKR 180 |
| | (SEQ ID | (SEQ ID |
| | NO: 5) | NO: 20) |
| PCV2b | 124 ILDDNFVT | 166 VLDSTID |
| (KR816332) | KATALTYDPY 141 | YFQPNNKR 180 |
| | (SEQ ID | (SEQ ID |
| | NO: 5) | NO: 20) |
| rPCV2-Vac | 124 ILDDNFVNK | 166 VLDSTIDY |
| | STALTYDPY 141 | FNPNNSR 180 |
| | (SEQ ID | (SEQ ID |
| | NO: 23) | NO: 24) |
| PCV2d | 124 ILDDNFVTK | 166 VLDRTIDY |
| (JX535296.1) | ANALTYDPY 141 | FQPNNKR 180 |
| | (SEQ ID | (SEQ ID |
| | NO: 25) | NO: 26) |

Bold residues-mismatches from the PCV2b vaccine (KR816332) backbone
Underlined residues-residues mutated in the rPCV2-Vac
Italicized residues-putative glycosylation sites (NetNGlyc 1.0 Server)

Insertion of a marker to differentiate vaccinated and infected (DIVA) pigs: As a high percentage of production swine are naturally infected with PCV2, the vaccine construct was designed to include a positive marker to enable DIVA capabilities. Neospora caninum is an apicomplexan parasite which has not been detected in pigs. A highly immunogenic segment of 18 amino acid length selected from the surface antigen-1 related sequence 2 (SRS2) protein (AAD04844.1) of N. caninum was selected following the in silico prediction of antigenicity (Lasergene 11, Protean 13, DNASTAR, USA). The selected sequence was subjected to a protein blast to rule out possible serological cross reactivity with other swine related proteins. Amino acids 324 QSSEKRDGEQVNKGKPP 348 (SEQ ID NO: 27) of the SRS2 protein, with an antigenicity index score of 1.7 was inserted into 5′ end of the capsid gene of the rPCV2-Vac construct described above as a separate transcriptional unit (FIGS. 6-7), using the Q5 mutagenesis kit (New England Biologicals, USA), according to the manufacturer's instructions.

Preparation of PCV2 virus cultures: The vaccine and challenge virus cultures, as well as the virus cultures required for the virus neutralization assay were prepared by transfection of PK-15 cells with some modifications. Briefly, the PCV2 genome was excised from the shuttle plasmid by restriction digestion and re-circularized with DNA ligase, unless dimerized infectious clones were available. For transfection, 12 μg of viral genomic DNA or plasmids containing the dimerized infectious clones were diluted in Opti-MEM, mixed with 36 μl of TransIT-2020 (Minis Bio, USA) and incubated at room temperature for 30 mins. After the incubation period, the mixture was overlaid on cell culture flasks (25 cm², Corning, USA) containing 50% confluent monolayers of PK-15 cells and incubated at 37° C. in a CO₂ incubator for 3 h, followed by addition of Dulbecco's Modified Eagle's Medium (DMEM) with 2% fetal bovine serum and 1× penicillin streptomycin. The flasks were frozen and thawed 3 times after 72 h of incubation. The rescued viruses were titrated by the TCID₅₀ method. The stock cultures were stored at −80° C. until used.

Immunofluorescence assay: As PCV2 does not produce cytopathic effects, replication of the PCV2 strains was visualized by IFA as previously described. Briefly, 50% confluent PK-15 monolayers grown in 8 well chamber slides were either transfected as described above or infected with the virus cultures. After 72 hrs of incubation in a CO₂ incubator, the cells were fixed with a 1:1 mixture of methanol: acetone. The fixed cell sheets were stained with a PCV2 specific monoclonal antibody (Rural Technologies, USA) or Neospora caninum specific mouse polyclonal antibody, followed by detection with a FITC-conjugated secondary antibody (KPL, USA), and counter-staining with DAPI (Life Technologies, USA). The stained cells were evaluated for apple green nuclear fluorescence indicative of PCV2 replication or expression of the SRS2 DIVA tag (FIGS. 4A-D).

In vitro vaccine stability: The rPCV2-Vac cultures rescued by transfection of PK-15 cells were serially passaged three times in PK-15 cells. Virus titers were compared against the wildtype virus. The construct was sequenced to verify the stability of the mutations.

Vaccination and challenge of piglets: All procedures pertaining to animal experimentation were carried out with the approval and oversight of the Institutional Animal Care and Use Committee (IACUC) and Institutional Biosafety Committee (IBC) regulations of N. Dakota (NDSU) and S. Dakota State Universities (SDSU). Twenty-seven, 3-4-week-old piglets which were serologically and PCR negative for PCV2 and other major swine pathogens such as PRRSV, SIV and Mycoplasma sp. were divided into 3 groups of 9 pigs each. Group I was administrated PBS, group II were administered a commercial, inactivated PCV2 vaccine as per label instructions (2 ml, intramuscular), and group III were inoculated with the rPCV2-Vac at 10⁴ TCID₅₀/ml, 2 ml intramuscular and 2 ml intranasally. Although the exact details regarding the antigen dose, formulation, and adjuvants present in the commercial vaccine are not publicly available, a commercial vaccine was selected as a control to represent current industry standards. On day 28 post vaccination (DPV) or day 0 post-challenge (DPC), all study animals were challenged with a heterologous PCV2d strain at 10⁴TCID₅₀, 2 ml intramuscular and 2 ml intranasally. Pigs were monitored daily for signs of porcine circovirus associated diseases (PCVAD) such as wasting, respiratory distress, jaundice, inappetence, or diarrhea. Body weights were assessed on DPC 0, 9, and 21. Serum samples were collected on day 0, and every 2 weeks thereafter to assess Ab responses. All animals were humanely euthanized on DPC 21 for evaluation of pathological lesions as described below.

Anti-PCV2 IgG responses: The measurement of binding IgG responses to PCV2 in vaccinated pigs was achieved with a commercial PCV2 ELISA kit (Ingezim Circovirus IgG kit, Ingenasa, Madrid, Spain), at the Iowa State University Veterinary Diagnostic Laboratory, following their standard operating procedures and the manufacturer's instructions. Signal to positive control (S/P) ratios produced as the assay output were used for further analysis of the data.

Virus neutralizing antibody responses: Functional antibody responses against the homologous PCV2b subtype and heterologous PCV2a and PCV2d subtypes were measured by a rapid fluorescence focus neutralization (FFN) assay, essentially as described before, except that the virus cultures were adjusted to 30-40 fluorescent focus units (FFU)/100 μl for consistent enumeration. Virus replication was assessed by an IFA, as described above. Four replicate values of the DPV 28 sera were obtained and used for analysis. The titers were expressed as the % reduction in viral replication compared to the virus only control, which was not treated with serum (FIG. 8).

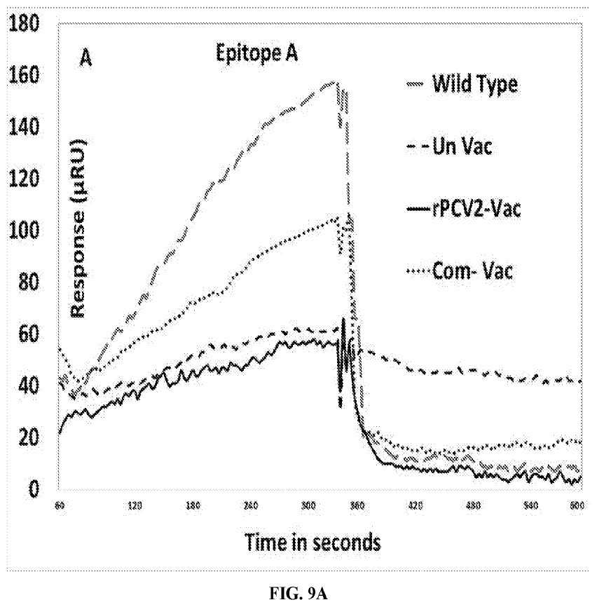

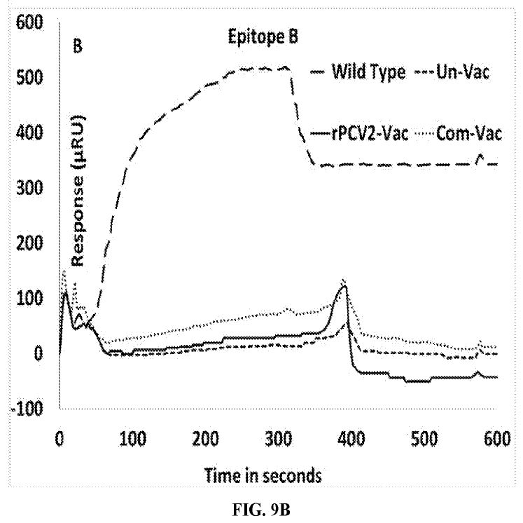

Antibody responses to the mutated epitopes: The abrogation of the immunodominant Ab response to the selected epitopes in vaccinated pigs was assessed by surface plasmon resonance on a Reichert SR7500DC instrument (Reichert Technologies, USA). Biotinylated peptides encoding the wildtype peptide sequences of epitopes A and B, as described above, were commercially synthesized (Biomatik, USA). Pooled sera collected at DPV 2S from the three treatment groups and from PCV2b infected pigs were used to purify IgG using a commercial kit (Melon gel IgG purification kit, Thermo Fisher, USA). The biotinylated peptides were immobilized on streptavidin coated carboxymethyl dextran sensor chips (Reichert Technologies, USA) by injecting 0.16 μg/μl peptide solution over the sensor chip at a flow rate of 25 μl/min. After an increase of about 300 μRU was observed, indicating immobilization of each peptide had occurred, the purified IgGs for the experimental groups were injected over the flow cells at a concentration of 20 μM in phosphate buffered saline with 0.005% Tween 20(PBST), at a flow rate of 25 μl/min for 240 secs. Binding of the IgGs to the peptides was assessed by the response in μ response units (μRU) (FIGS. 9A-B).

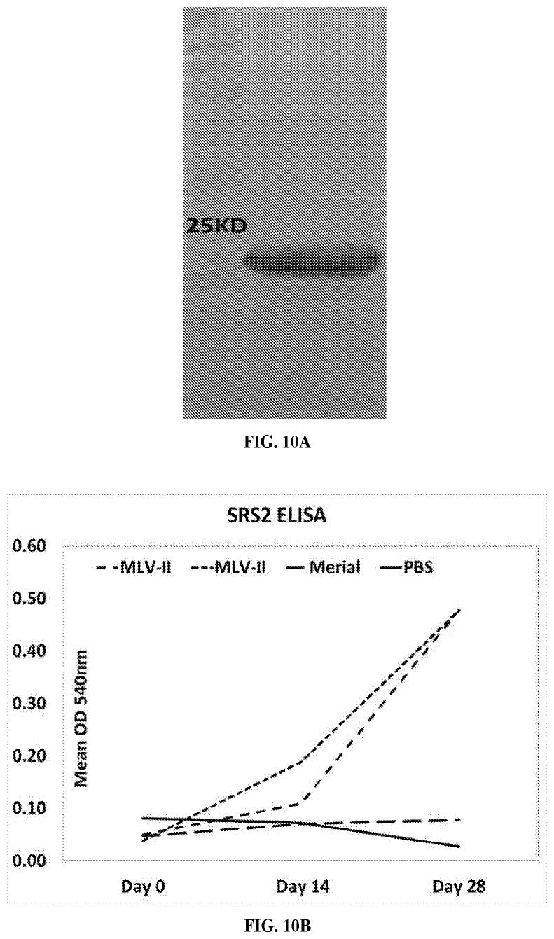

Antibody responses to the DIVA marker: The selected peptide from the N. caninum SRS2 protein was cloned into a bacterial expression vector (pETSumo Thermo Fisher Scientific, USA) using the Q5 site directed mutagenesis kit (New England Biologicals, USA). The protein was expressed with a HIS tag and purified by nickel affinity chromatography (His-spin protein miniprep, Zymo research, USA), following the manufacturer's instructions. The identity of the purified protein was verified by Western blotting with an anti-HIS tag specific monoclonal Ab (FIG. 10A). The purified protein was used to coat ELISA plates, followed by washing with PBST and blocking (General block with 2% BSA, Immuno Chemistry Technologies, USA) for 2 h at 37° C. The blocked plates were washed with PBST. A 1:50 dilution of the test anti-sera was diluted in PBS with 2% BSA, added to the wells and incubated for 2 h. The plates were then reacted with a 1:5000 dilution of anti-swine IgG conjugated to HPO (KPL, USA), followed by addition of TMB substrate. The reaction was stopped with 1M HCl and measurement of antibody responses to the SRS2 DIVA peptide in vaccinated pigs was measured by a SRS2 peptide specific ELISA (FIG. 10B).

Measurement of vaccine viral replication by qPCR: Replication of the rPCV2-Vac virus following immunization was quantified by a TaqMan quantitative PCR (qPCR), using a SRS2 marker specific primer and probe combination, and serum collected on DPV 0, 14, and 28. Samples were assessed in duplicate. Viral DNA was extracted using the QiaAmp DNA mini Kit (Qiagen, USA) according to manufacturer's instruction. Primer pairs with sequences of 5′-AAGTGGGAGGTTTGCCTTTGT-3′ (SEQ ID NO: 28) and 5′-ATGGCCCAATCCTCGGAGAA-3′ (SEQ ID NO: 29) and a probe with a sequence of 5′-TACCTGTTCCCCGTCGCGT-3′ (SEQ ID NO: 30) were used. Briefly, 2.0 μl of extracted DNA, 0.4 μM of primers, 0.1 μM probe, and a Tm of 67° C. were used in combination with the QuantiFast Probe PCR Kit (Qiagen, USA) and cycled in a qPCR thermocycler (CFX96 Touch, Bio-Rad, USA). The obtained Ct values were converted to log copy numbers using a standard curve generated with plasmid DNA encoding the SRS2 DIVA marker. The specificity of the assay was evaluated using the infectious clones for the wildtype PCV2b and heterologous PCV2a and PCV2d. The lowest limit of detection of the assay was 2000 genomic copies per ml of serum.

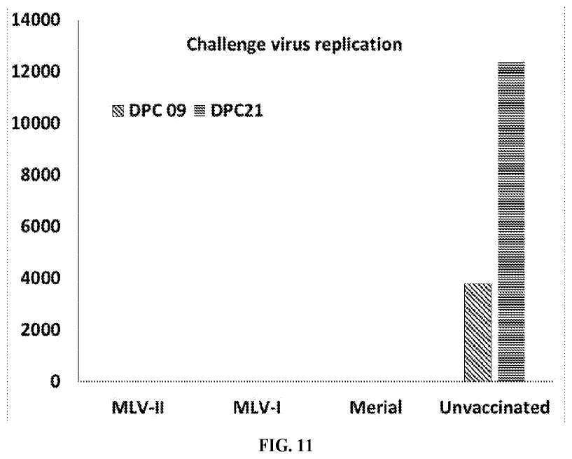

Detection of challenge viral replication: A qPCR assay which is specific to the PCV2d subtype was designed after analysis of PCV2a, PCV2b and PCV2d sequences to identify regions unique to PCV2d (FIG. 6). The sequences of the primers used were 5′-GGCCTACATGGTCTACATTTCCAGT-3′ (SEQ ID NO: 31) and 5′-GGTACTTTACCCCGAAACCTGTC-3′ (SEQ ID NO: 32), and the probe sequence was 5′-TGGGTTGGAAGTAATCGATTGTCCTATCA-3′ (SEQ ID NO: 33) (Biosearch Technologies, USA). The specificity of the assay for PCV2d was evaluated by testing for the absence of detection with PCV2a and PCV2b. A standard curve was generated using cloned PCV2d genomic DNA and the lowest limit of reliable detection determined as 3000 genomic copies per ml of serum. To quantify the challenge virus loads in serum, post-challenge sera collected at DPC 9 and DPC 21 were assessed essentially as described above (FIG. 11).

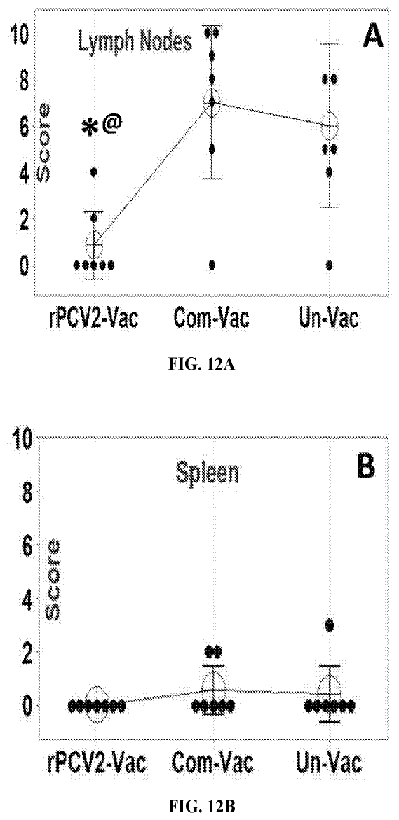

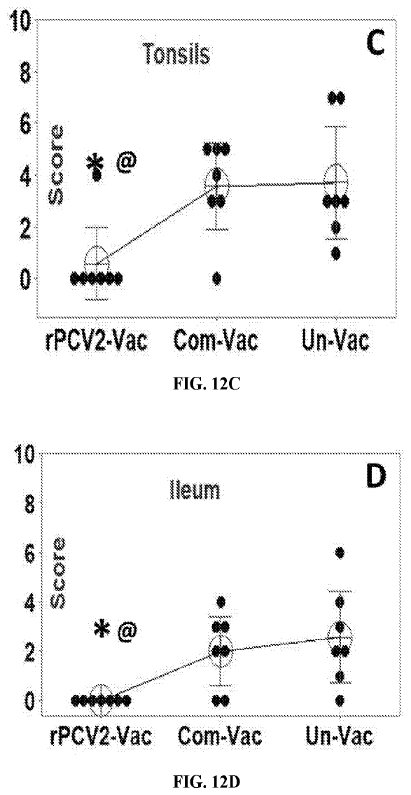

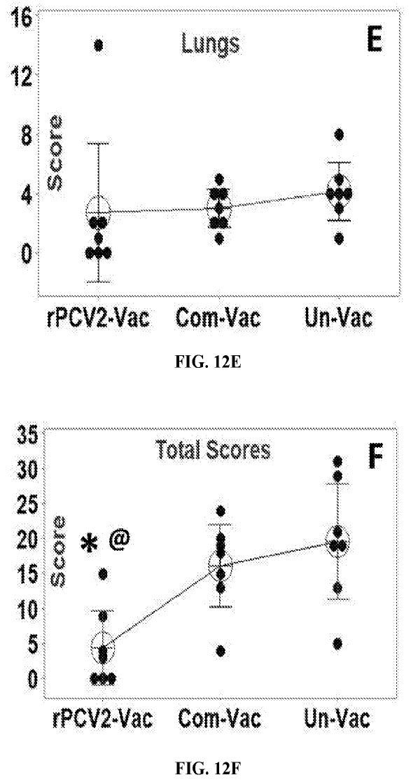

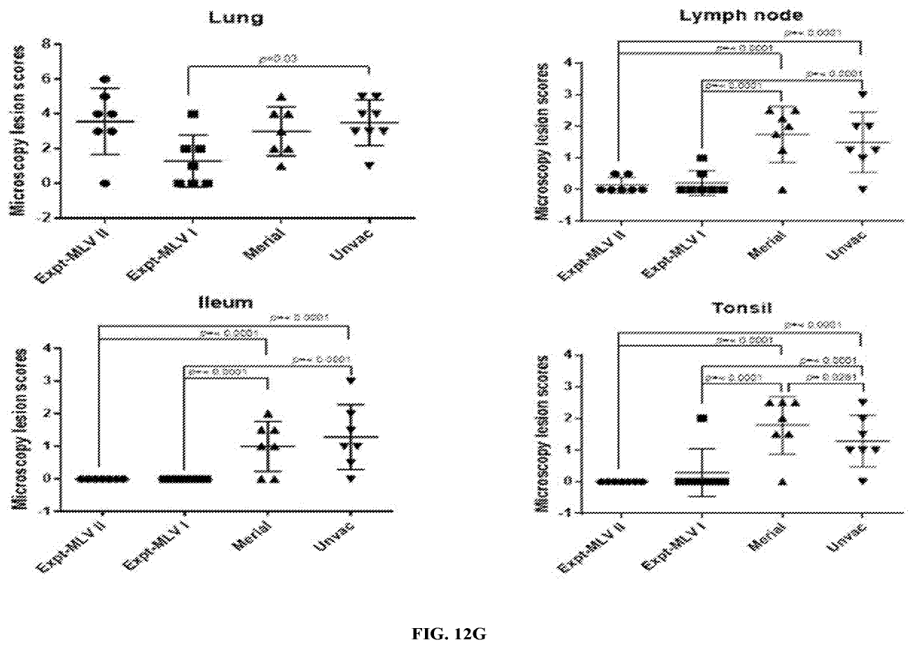

Assessment of pathological lesions: Evaluation of tissue pathology was carried out as described previously. Macroscopic evaluation of the major organs for gross lesions in the major organs was conducted by assessing lungs for the presence of lesions scored as the percentage of lung parenchyma affected from 1-100%. Inguinal lymph node enlargement was scored from 0-3, where 0 was no enlargement, 1, 2 and 3 were two, three or four times the normal size. Sections of the major organs including the lung, liver, kidney, spleen ileum, tonsils, tracheobronchial and mesenteric lymph nodes were fixed in 10% buffered formalin for 48 h and then transferred to 70% ethanol for sectioning. Slides were examined by hematoxylin and eosin (H&E) staining for microscopic lesions and immunohistochemistry (IHC) to detect viral antigen, following the standard operating procedures of the Iowa State University Veterinary Diagnostic Laboratory. The slides were assigned scores ranging from 1-4 in a blinded fashion by a board-certified veterinary pathologist as follows; 1=single follicle or focus staining, 2=rare to scattered staining, 3=moderate staining, 4=strong widespread staining (FIGS. 12A-F).

Statistical analysis: A significance level of p<0.05 was used for all statistical analysis. Analysis was conducted using the Minitab 19 software (Minitab, State College USA) or Microsoft excel. Where data was not normally distributed, non-parametric analysis was used. Serological and qPCR data were analyzed by a Student's T test. The lesion scores and body weight data were analyzed by the Mann Whitney U test. The consolidated values, statistical significance and standard deviation are represented in the figures.

Results:

The rPCV2-Vac was successfully rescued and expressed the DIVA peptide: The reverse genetics approaches were used to mutate the selected immunodominant linear B cell epitopes in the PCV2 capsid protein enable the successful rescue of the recombinant rPCV2 Vac virus. There were no significant differences between the titers of the wildtype PCV2b 41513 and the rPCV2 Vac virus cultures generated by transfection with the respective infectious clones (FIG. 4A). Introduction of the mutations did not affect detection of the recombinant PCV2 virus by polyclonal antibodies. Expression of the DIVA peptide was clearly detected by a Neospora caninum specific antibody (FIG. 4B).

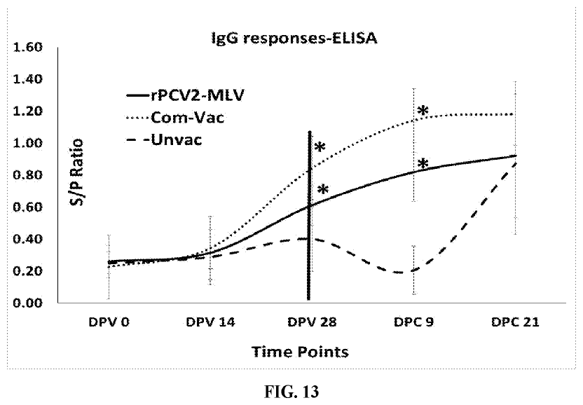

The rPCV2-Vac induces binding antibody responses in vaccinated pigs: Measurement of anti-PCV2 IgG responses in the study animals using a commercial PCV2 ELISA kit showed an increase in titers after 14 DPV in both the vaccine groups, with the differences between rPCV2-Vac and unvaccinated control group being significantly different at DPV 28 and DPC 09. Although a direct comparison between rPCV2-Vac and the commercial control cannot be drawn due to differences in vaccine formulation, the magnitude of the IgG response to the commercial vaccine remained consistently higher than that of the rPCV2-Vac. As expected, antibody responses in the unvaccinated controls remained low until DPC 9, after which significant differences were not noted between the groups at DPC 21 (FIG. 13).

The rPCV2-Vac elicits broad virus neutralization responses: To determine if the mutation of immunodominant, non-protective epitopes would improve the cross-neutralization response to heterosubtypic strains, virus neutralizing responses were measured against the homologous PCV2b subtype as well as heterologous PCV2a and PCV2d subtypes using a rapid fluorescence focus reduction assay. Both MLV-I and MLV-II were highly effective in neutralizing all three PCV2 subtypes tested. Despite the fact that the commercial vaccine has an adjuvant and has undergone extensive dose optimization, neutralization responses elicited by the rPCV2-Vac against the PCV2a subtype was comparable in kinetics and magnitude to that of the commercial vaccine, which contains the PCV2a capsid antigen. Similarly, neutralizing responses against the currently predominant PCV2d subtype in the rPCV2-Vac group were higher than that of commercial vaccine by DPV14, with the difference becoming statistically significant at DPV28. As expected, neutralizing responses elicited by the rPCV2-Vac against its homologous PCV2b strain were robust. However, the commercial vaccine was significantly less effective than rPCV2-Vac in neutralizing PCV2b. Overall, the data supports the conclusion that rPCV2-Vac was more effective in neutralizing heterologous subtypes than the PCV2a based commercial vaccine (FIG. 8).

Mutation abrogates antibody responses to the selected epitopes. As expected, antibody responses to epitope A and B were not detected in the serum of rPCV2-Vac immunized pigs by a qualitative SPR analysis, while the responses in pigs infected with the wildtype virus were strong. For epitope 1A, the response in pigs administered the rPCV2-Vac was similar to that of the unvaccinated pigs. The response in the pigs administered the commercial vaccine was of a lesser magnitude than that of the pigs infected with the wildtype virus. In the case of epitope B, strong responses were noted pigs infected with the wildtype virus as expected, but the differences between the other three groups were not significant (FIGS. 9A-B).

Vaccinated pigs mount DIVA tag specific Ab responses: Assessment of the antibody responses to the DIVA marker by an ELISA specific to the peptide selected from the N. caninum SRS2 protein showed that pigs in the vaccinated groups mounted detectable Abs responses to the DIVA marker by DPV14, with the magnitude of the responses increasing until DPV 28. As expected, the unvaccinated pigs and pigs administered the commercial vaccine did not mount significant antibody responses to the DIVA marker (FIG. 10B).

Vaccination protects against challenge viral replication: Replication of the heterologous PCV2d challenge virus was not detected in either of the vaccine groups at DPC 9 or DPC 21. As expected, robust challenge viral replication was detected in the unvaccinated pigs, with the titers increasing by about 1 log between day 9 and day 21 post-challenge. In contrast, challenge viral replication was not detected in any of the vaccinated pigs, including those administered the commercial vaccine, indicating that the experimental vaccine induced sterilizing immunity. The values for both vaccine groups were significantly different from the unvaccinated control group at both the time points tested (FIG. 11).

Protection against gross and histological lesions: Except for the lungs, gross lesions were not observed in any of the other major organs for all experimentally challenged pigs (FIGS. 12A-G). For the lymph nodes, the microscopic lesion scores (consisting of the sum of the H&E and IHC scores) were significantly lower for the rPCV2-Vac group than those of the commercial vaccine group and the unvaccinated group (FIG. 12A) with only 2 out of 7 pigs showed mild changes while 6 of 7 pigs in the control groups showed histiocytic infiltration and lymphoid depletion. Microscopic lesions were not detected in the spleen, liver, and heart (FIG. 12B). The microscopic lesion scores of the ileum and tonsils (FIGS. 12C-D) of the rPCV2-Vac group were also significantly lower than that of the control groups. The pulmonary lesion scores in the rPCV2-Vac group were lower than that of the controls but the difference was not statistically significant (FIG. 12E). The overall lesion scores for the rPCV2-Vac was highly significantly different from the control groups (FIG. 12F), while the scores of the commercial vaccine group was similar to that of the unvaccinated group. Lung microscopic lesions were comparable between MLV-II and the commercial vaccine while they were lower in MLV-I vaccinated animals (FIG. 12G). No viral antigen was detected in the lung, indicating that the lesions were resolving after viral clearance in both MLV's.

Vaccination protects against weight loss due to challenge: As is commonly encountered in experimental models, severe clinical signs of PCVAD were not observed in any of the experimental groups during the 21 days post-challenge observation period. However, the post-challenge weight gain in both vaccination groups were significantly higher than the unvaccinated control group at DPC 21, but not DPC 14. There were no significant differences between the two vaccine groups during the post-challenge observation period.

The rPCV2-Vac is safe and stable: In contrast to wildtype PCV2 viruses, which can be easily detected by qPCR by DPC 9 (FIG. 11), viremia due to the rPCV2-Vac virus was not detected by the SRS2 DIVA tag-specific qPCR assay in the sera of any of the vaccinated pigs at DPV14. The rPCV2-Vac virus was detected at low levels in the serum of only in 1 out of 9 pigs at DPV 28, indicating that the rPCV2-MLV was attenuated in vivo. Sequencing of the rPCV2-Vac genome from the viremic pig confirmed the presence of the mutations in the 2 epitopes and the presence of the DIVA tag, indicating the vaccine remained stable in the host. Significant gross or microscopic lesions were not observed in the pigs sacrificed prior to challenge (2 pigs per group) to assess vaccine safety. There were no significant differences in the lesion scores between the experimental groups, indicating that the rPCV2-Vac was safe. Similarly, sequencing of the rPCV2-Vac genome after 3 passages in cell culture showed that the mutated and inserted sequences were intact, indicating that the vaccine was genetically stable in vitro.

Discussion

The phenomenon of “original antigenic sin” or ability to elicit memory responses to antigens and specific epitopes is critical to the success of vaccination. On the other hand, the preferential clonal expansion to immuno-dominant but non-protective epitopes encountered by the host on challenge, coupled with minor sequence variation leading to escape variants, is an elegant immuno-subversion strategy the present inventors termed “deceptive imprinting.” Strategies to counter deceptive imprinting in vaccine design include “dampening” the response to the immuno-dominant, non-protective epitopes. The immune refocusing strategy has been successfully applied to several viruses such as human immunodeficiency virus (HIV), influenza, and dengue virus, among others. Unlike structurally complex pathogens, where protection is mediated by multiple antigens, the requirement for a single protective antigen makes PCV2 both a simple and elegant model for studying the effects of immunodominance on vaccine design. This Example explored the hypothesis that alteration of the immunodominance properties of the PCV2 capsid protein will result in the improvement of vaccine efficacy.

The PCV2 capsid protein contains four major immunodominant regions. Within these regions, 4 putative immunodominant, non-protective linear B cell epitopes have been identified. As the PCV2 capsid protein is relatively small (233 amino acids), and incapable of tolerating large sequence changes, only two of the identified decoy epitopes were selected for mutation in this Example. It was previously demonstrated that mutation of an immunodominant HIV-1 epitope located in proximity to a neutralizing epitope can direct the response towards the neutralizing epitopes, possibly due to alteration of steric constraints. As both epitope A and B were flanked by putative neutralizing epitopes they were selected for analysis. To minimize the risk of introducing lethal mutations, the present inventors elected not to delete residues but rather replace them with other residues with a low penalty score on a point accepted mutation (PAM) matrix and were able to successfully rescue the recombinant virus harboring mutations in the selected epitopes (FIGS. 4A-D).

As anticipated, the introduced changes to the amino acid sequences of the PCV2 capsid protein resulted in the loss of immunodominance of epitope A and B as assessed by SPR (FIGS. 9A-B). With the loss of immunodominance, an overall reduction in the magnitude of the binding antibody response was also noted (FIG. 13), which corresponded with an improved performance of the developed vaccine. As paratopes which bind rapidly to their epitopes receive stronger stimulatory signals and can influence the magnitude of clonal expansion during the affinity maturation stage, an assessment of the affinity kinetics of the Abs generated in this Example to their cognate peptides could not be carried out due to a shortage of samples and only a qualitative measurement was obtained by SPR (FIGS. 9A-B). Interestingly, antibody responses to epitope B were not detected in pigs administered the commercial PCV2 vaccine. It has been previously suggested that vaccination with fully assembled viral particles does not induce strong Ab responses to epitope B while vaccination with monomers of the subunit does. Further, MHC-II processing for the same antigen is known to differ between endogenous and exogenous antigens which may be introduced by infection or vaccination respectively. A limitation of this study is that only linear epitopes were targeted.

Several other factors such as glycosylation, hypervariability, and proximity to MHC-II epitopes or other neutralizing epitopes could also potentially influence the outcomes of this Example. While a detailed experimental characterization of the above listed parameters is not within the scope of the Example, they are discussed below. Hyper-glycosylation is a strategy which has been previously used to dampen the Ab response to immunodominant epitopes. While not the primary strategy targeted in this Example, the replacement of a threonine (T) with an asparagine (N) residue in epitope A resulted in the introduction of a putative N-linked glycosylation sequon (N×S) (Table 3). Epitope B naturally contained a predicted N-linked glycosylation site (Table 3), and was not altered for glycosylation properties. As immunodominance is influenced by the successful competition for the recruitment of antigen specific T cells in early infection, the presence of a helper T cell epitopes overlapping or adjacent to a B cell epitope can influence the strength of the Ab response elicited. Epitope A contained a predicted (Propred MHC-II server), but non-conserved, MHC-II epitope 124 ILDDNFVT 131 (SEQ ID NO: 34) in the rPCV2-Vac backbone, which was altered by the mutation of the residue T to an N. Two conserved, predicted MHC-II epitopes, 161 FTPKPVL 167 (SEQ ID NO: 35) and 174 FQPNNKRNQL 184 (SEQ ID NO: 36) overlapped with epitope B. The second predicted MHC-II epitope within epitope B was also altered by the mutations introduced. It is possible that mutation of these T helper epitopes could have enhanced the loss of immunodominance of Epitopes A and B.

Hypervariability is a common property of decoy epitopes, and is an effective immuno-subversion mechanism. However, epitopes A and B were conserved between the first discovered PCV2a and PCV2b subtypes (Table 3, FIG. 6). Only residue 131 in epitope A and residue 169 in epitope B varied between the newly evolved PCV2d challenge strain and the previously existing PCV2a and 2b subtypes (Table 3, FIG. 6). For influenza, it has been suggested that the reduced vaccine efficacy observed for the H3N2 component of the polyvalent vaccine could result from the reinforcement of persistent and preferential strain specific memory (deceptive imprinting) to the H1 subtype and B type by annual vaccination, leading to competition between the polyvalent antigens. Therefore, prior exposure to the unmodified epitopes A and B by infection with PCV2a or 2b, or by vaccination, could diminish protection against the newly evolved PCV2d subtype in the field. While direct comparisons of the rPCV2-Vac to the commercial control vaccine are avoided as the commercial vaccine is extensively standardized for optimal dosage and contains an adjuvant, in this study, the rPCV2-Vac was significantly more effective at inducing neutralizing Ab responses against the heterologous PCV2d subtype (FIG. 8). However, the field situations the level of cross-neutralization and protection between the three contemporary PCV2 subtypes is likely sufficient for controlling clinical manifestations of PCVAD, but not preventing viral evolution and emergence of new subtypes.

The lack of challenge viral replication resulting in sterilizating immunity (FIG. 11), and the broadened virus neutralization responses elicited by vaccination with rPCV2-Vac (FIG. 8) correlated with the significant reduction in tissue pathology caused by early challenge viral replication and localization to the sites of predilection (FIGS. 12A-G). The reduced lesion scores in lymphoid organs, which are the primary sites of predilection for PCV2, indicate the rPCV2-Vac was highly effective in curtailing local infection as well as systemic dissemination. With the reasonably strong performance of current PCV2 vaccines in the field, the availability of an enhanced vaccine could pave the way for the eventual eradication of the virus. Successful disease eradication efforts in veterinary medicine typically employ a stamping out strategy, wherein infected animals can be differentiated from vaccinated animals using serological assays and then removed from the herd in a systematic manner. The DIVA capability of the rPCV2-Vac (FIGS. 5A-D and 10B) anticipates the need for a PCV2 DIVA vaccine to support eventual eradication efforts. With additional dose optimization and possible commercialization, the improved efficacy parameters of the rPCV2-Vac could reduce or eliminate the emergence of new PCV2 subtypes, and significantly advance current control measures for PCV2.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

(1) Afghah, Z., B. Webb, X. J. Meng and S. Ramamoorthy (2017). “Ten years of PCV2 vaccines and vaccination: Is eradication a possibility?” Vet Microbiol 206: 21-28.
(2) Agarwal, A. and K. V. Rao (1997). “B cell responses to a peptide epitope: III. Differential T helper cell thresholds in recruitment of B cell fine specificities.” J Immunol 159(3): 1077-1085.
(3) Barker, W. C. and M. O. Dayhoff (1979). “Evolution of homologous physiological mechanisms based on protein sequence data.” Comp Biochem Physiol B 62(1): 1-5.
(4) Barnett, S. W., S. Lu, I. Srivastava, S. Cherpelis, A. Gettie, J. Blanchard, S. Wang, I. Mboudjeka, L. Leung, Y. Lian, A. Fong, C. Buckner, A. Ly, S. Hilt, J. Ulmer, C. T. Wild, J. R. Mascola and L. Stamatatos (2001). “The ability of an oligomeric human immunodeficiency virus type 1 (HIV-1) envelope antigen to elicit neutralizing antibodies against primary HIV-1 isolates is improved following partial deletion of the second hypervariable region.” J Virol 75(12): 5526-5540.
(5) Bonifaz, L. C., S. Arzate and J. Moreno (1999). “Endogenous and exogenous forms of the same antigen are processed from different pools to bind MHC class II molecules in endocytic compartments.” Eur J Immunol 29(1): 119-131.
(6) Constans, M., M. Ssemadaali, O. Kolyvushko and S. Ramamoorthy (2015). “Antigenic Determinants of Possible Vaccine Escape by Porcine Circovirus Subtype 2b Viruses.” Bioinform Biol Insights 9(Suppl 2): 1-12.
(7) Donahoe, S. L., S. A. Lindsay, M. Krockenberger, D. Phalen and J. Slapeta (2015). “A review of neosporosis and pathologic findings of Neospora caninum infection in wildlife.” Int J Parasitol Parasites Wildl 4(2): 216-238.
(8) Fenaux, M., P. G. Halbur, G. Haqshenas, R. Royer, P. Thomas, P. Nawagitgul, M. Gill, T. E. Toth and X. J. Meng (2002). “Cloned genomic DNA of type 2 porcine circovirus is infectious when injected directly into the liver and lymph nodes of pigs: characterization of clinical disease, virus distribution, and pathologic lesions.” J Virol 76(2): 541-551.
(9) Fenaux, M., T. Opriessnig, P. G. Halbur and X. J. Meng (2003). “Immunogenicity and pathogenicity of chimeric infectious DNA clones of pathogenic porcine circovirus type 2 (PCV2) and nonpathogenic PCV1 in weanling pigs.” J Virol 77(20): 11232-11243.
(10) Firth, C., M. A. Charleston, S. Duffy, B. Shapiro and E. C. Holmes (2009). “Insights into the evolutionary history of an emerging livestock pathogen: porcine circovirus 2.” J Virol 83(24): 12813-12821.
(11) Francis, M. J. (2018). “Recent Advances in Vaccine Technologies.” Vet Clin North Am Small Anim Pract 48(2): 231-241.
(12) Frei, J. C., A. S. Wirchnianski, J. Govero, O. Vergnolle, K. A. Dowd, T. C. Pierson, M. Kielian, M. E. Girvin, M. S. Diamond and J. R. Lai (2018). “Engineered Dengue Virus Domain III Proteins Elicit Cross-Neutralizing Antibody Responses in Mice.” J Virol 92(18).
(13) Garrity, R. R., G. Rimmelzwaan, A. Minassian, W. P. Tsai, G. Lin, J. J. de Jong, J. Goudsmit and P. L. Nara (1997). “Refocusing neutralizing antibody response by targeted dampening of an immunodominant epitope.” J Immunol 159(1): 279-289.
(14) Guo, L., Y. Lu, L. Huang, Y. Wei and C. Liu (2011). “Identification of a new antigen epitope in the nuclear localization signal region of porcine circovirus type 2 capsid protein.” Intervirology 54(3): 156-163.
(15) Huang, L. P., Y. H. Lu, Y. W. Wei, L. J. Guo and C. M. Liu (2011). “Identification of one critical amino acid that determines a conformational neutralizing epitope in the capsid protein of porcine circovirus type 2.” BMC Microbiol 11: 188.
(16) Ilha, M., P. Nara and S. Ramamoorthy (2020). “Early antibody responses map to non-protective, PCV2 capsid protein epitopes.” Virology 540: 23-29.
(17) Jeffs, S. A., C. Shotton, P. Balfe and J. A. McKeating (2002). “Truncated gp120 envelope glycoprotein of human immunodeficiency virus 1 elicits a broadly reactive neutralizing immune response.” J Gen Virol 83(Pt 11): 2723-2732.
(18) Jin, J., C. Park, S. H. Cho and J. Chung (2018). “The level of decoy epitope in PCV2 vaccine affects the neutralizing activity of sera in the immunized animals.” Biochem Biophys Res Commun 496(3): 846-851.
(19) Karuppannan, A. K. and T. Opriessnig (2017). “Porcine Circovirus Type 2 (PCV2) Vaccines in the Context of Current Molecular Epidemiology.” Viruses 9(5).
(20) Khayat, R., N. Brunn, J. A. Speir, J. M. Hardham, R. G. Ankenbauer, A. Schneemann and J. E. Johnson (2011). “The 2.3-angstrom structure of porcine circovirus 2.” J Virol 85(15): 7856-7862.
(21) Kittlesen, D. J., L. R. Brown, V. L. Braciale, J. P. Sambrook, M. J. Gething and T. J. Braciale (1993). “Presentation of newly synthesized glycoproteins to CD4+T lymphocytes. An analysis using influenza hemagglutinin transport mutants.” J Exp Med 177(4): 1021-1030.
(22) Kolyvushko, O., A. Rakibuzzaman, A. Pillatzki, B. Webb and S. Ramamoorthy (2019). “Efficacy of a Commercial PCV2a Vaccine with a Two-Dose Regimen Against PCV2d.” Vet Sci 6(3).
(23) Lee, H., E. H. Shim and S. You (2018). “Immunodominance hierarchy of influenza subtype-specific neutralizing antibody response as a hurdle to effectiveness of polyvalent vaccine.” Hum Vaccin Immunother 14(10): 2537-2539.
(24) Lekcharoensuk, P., I. Morozov, P. S. Paul, N. Thangthumniyom, W. Wajjawalku and X. J. Meng (2004). “Epitope mapping of the major capsid protein of type 2 porcine circovirus (PCV2) by using chimeric PCV1 and PCV2.” J Virol 78(15): 8135-8145.
(25) Lin, K., C. Wang, M. P. Murtaugh and S. Ramamoorthy (2011). “Multiplex method for simultaneous serological detection of porcine reproductive and respiratory syndrome virus and porcine circovirus type 2.” J Clin Microbiol 49(9): 3184-3190.
(26) Liu, J., L. Huang, Y. Wei, Q. Tang, D. Liu, Y. Wang, S. Li, L. Guo, H. Wu and C. Liu (2013). “Amino acid mutations in the capsid protein produce novel porcine circovirus type 2 neutralizing epitopes.” Vet Microbiol 165(3-4): 260-267.
(27) Madson, D. M., S. Ramamoorthy, C. Kuster, N. Pal, X. J. Meng, P. G. Halbur and T. Opriessnig (2009). “Infectivity of porcine circovirus type 2 DNA in semen from experimentally-infected boars.” Vet Res 40(1): 10.
(28) Mahe, D., P. Blanchard, C. Truong, C. Arnauld, P. Le Cann, R. Cariolet, F. Madec, E. Albina and A. Jestin (2000). “Differential recognition of ORF2 protein from type 1 and type 2 porcine circoviruses and identification of immunorelevant epitopes.” J Gen Virol 81(Pt 7): 1815-1824.
(29) Meerts, P., G. Misinzo, D. Lefebvre, J. Nielsen, A. Botner, C. S. Kristensen and H. J. Nauwynck (2006). “Correlation between the presence of neutralizing antibodies against porcine circovirus 2 (PCV2) and protection against replication of the virus and development of PCV2-associated disease.” BMC Vet Res 2: 6.
(30) Misinzo, G., P. L. Delputte, P. Meerts, D. J. Lefebvre and H. J. Nauwynck (2006). “Porcine circovirus 2 uses heparan sulfate and chondroitin sulfate B glycosaminoglycans as receptors for its attachment to host cells.” J Virol 80(7): 3487-3494.
(31) Nara, P. L. (1999). “Deceptive imprinting: insights into mechanisms of immune evasion and vaccine development.” Adv Vet Med 41: 115-134.
(32) Nara, P. L. and R. Garrity (1998). “Deceptive imprinting: a cosmopolitan strategy for complicating vaccination.” Vaccine 16(19): 1780-1787.
(33) Nara, P. L., G. J. Tobin, A. R. Chaudhuri, J. D. Trujillo, G. Lin, M. W. Cho, S. A. Levin, W. Ndifon and N. S. Wingreen (2010). “How can vaccines against influenza and other viral diseases be made more effective?” PLoS Biol 8(12): e1000571.
(34) Nayak, B. P., A. Agarwal, P. Nakra and K. V. Rao (1999). “B cell responses to a peptide epitope. VIII. Immune complex-mediated regulation of memory B cell generation within germinal centers.” J Immunol 163(3): 1371-1381.
(35) Opriessnig, T., S. Ramamoorthy, D. M. Madson, A. R. Patterson, N. Pal, S. Carman, X. J. Meng and P. G. Halbur (2008). “Differences in virulence among porcine circovirus type 2 isolates are unrelated to cluster type 2a or 2b and prior infection provides heterologous protection.” J Gen Virol 89(Pt 10): 2482-2491.
(36) Patterson, A. R., J. Johnson, S. Ramamoorthy, X. J. Meng, P. G. Halbur and T. Opriessnig (2008). “Comparison of three enzyme-linked immunosorbent assays to detect Porcine circovirus-2 (PCV-2)-specific antibodies after vaccination or inoculation of pigs with distinct PCV-1 or PCV-2 isolates.” J Vet Diagn Invest 20(6): 744-751.
(37) Patterson, A. R., S. Ramamoorthy, D. M. Madson, X. J. Meng, P. G. Halbur and T. Opriessnig (2011). “Shedding and infection dynamics of porcine circovirus type 2 (PCV2) after experimental infection.” Vet Microbiol 149(1-2): 91-98.
(38) Pogranichnyy, R. M., K. J. Yoon, P. A. Harms, S. L. Swenson, J. J. Zimmerman and S. D. Sorden (2000). “Characterization of immune response of young pigs to porcine circovirus type 2 infection.” Viral Immunol 13(2): 143-153.
(39) Rajewsky, K. (1996). “Clonal selection and learning in the antibody system.” Nature 381(6585): 751-758.
(40) Ramamoorthy, S., F. F. Huang, Y. W. Huang and X. J. Meng (2009). “Interferon-mediated enhancement of in vitro replication of porcine circovirus type 2 is influenced by an interferon-stimulated response element in the PCV2 genome.” Virus Res 145(2): 236-243.
(41) Ramamoorthy, S. and X. J. Meng (2009). “Porcine circoviruses: a minuscule yet mammoth paradox.” Anim Health Res Rev 10(1): 1-20.
(42) Ramamoorthy, S., T. Opriessnig, N. Pal, F. F. Huang and X. J. Meng (2011). “Effect of an interferon-stimulated response element (ISRE) mutant of porcine circovirus type 2 (PCV2) on PCV2-induced pathological lesions in a porcine reproductive and respiratory syndrome virus (PRRSV) co-infection model.” Vet Microbiol 147(1-2): 49-58.
(43) Ramamoorthy, S., N. Sanakkayala, R. Vemulapalli, R. B. Duncan, D. S. Lindsay, G. S. Schurig, S. M. Boyle, R. Kasimanickam and N. Sriranganathan (2007). “Prevention of lethal experimental infection of C57BL/6 mice by vaccination with Brucella abortus strain RB51 expressing Neospora caninum antigens.” Int J Parasitol 37(13): 1521-1529.
(44) Ramamoorthy, S., N. Sanakkayala, R. Vemulapalli, N. Jain, D. S. Lindsay, G. S. Schurig, S. M. Boyle and N. Sriranganathan (2007). “Prevention of vertical transmission of Neospora caninum in C57BL/6 mice vaccinated with Brucella abortus strain RB51 expressing N. caninum protective antigens.” Int J Parasitol 37(13): 1531-1538.
(45) Reynolds, C. R., S. A. Islam and M. J. E. Sternberg (2018). “EzMol: A Web Server Wizard for the Rapid Visualization and Image Production of Protein and Nucleic Acid Structures.” J Mol Biol 430(15): 2244-2248.
(46) Saha, D., L. Huang, E. Bussalleu, D. J. Lefebvre, M. Fort, J. Van Doorsselaere and H. J. Nauwynck (2012). “Antigenic subtyping and epitopes' competition analysis of porcine circovirus type 2 using monoclonal antibodies.” Vet Microbiol 157(1-2): 13-22.
(47) Shang, S. B., Y. L. Jin, X. T. Jiang, J. Y. Zhou, X. Zhang, G. Xing, J. L. He and Y. Yan (2009). “Fine mapping of antigenic epitopes on capsid proteins of porcine circovirus, and antigenic phenotype of porcine circovirus type 2.” Mol Immunol 46(3): 327-334.
(48) Schwartz, R. M. and M. O. Dayhoff (1979). “Protein and nucleic Acid sequence data and phylogeny.” Science 205(4410): 1038-1039.
(49) Singh, H. and G. P. Raghava (2001). “ProPred: prediction of HLA-DR binding sites.” Bioinformatics 17(12): 1236-1237.
(50) Ssemadaali, M. A., M. Ilha and S. Ramamoorthy (2015). “Genetic diversity of porcine circovirus type 2 and implications for detection and control.” Res Vet Sci 103: 179-186.
(51) Tobin, G. J., J. D. Trujillo, R. V. Bushnell, G. Lin, A. R. Chaudhuri, J. Long, J. Barrera, L. Pena, M. J. Grubman and P. L. Nara (2008). “Deceptive imprinting and immune refocusing in vaccine design.” Vaccine 26(49): 6189-6199.
(52) Trible, B. R., M. Kerrigan, N. Crossland, M. Potter, K. Faaberg, R. Hesse and R. R. Rowland (2011). “Antibody recognition of porcine circovirus type 2 capsid protein epitopes after vaccination, infection, and disease.” Clin Vaccine Immunol 18(5): 749-757.
(53) Trible, B. R., A. Ramirez, A. Suddith, A. Fuller, M. Kerrigan, R. Hesse, J. Nietfeld, B. Guo, E. Thacker and R. R. Rowland (2012). “Antibody responses following vaccination versus infection in a porcine circovirus-type 2 (PCV2) disease model show distinct differences in virus neutralization and epitope recognition.” Vaccine 30(27): 4079-4085.
(54) Trible, B. R., A. W. Suddith, M. A. Kerrigan, A. G. Cino-Ozuna, R. A. Hesse and R. R. Rowland (2012). “Recognition of the different structural forms of the capsid protein determines the outcome following infection with porcine circovirus type 2.” J Virol 86(24): 13508-13514.
(55) Trujillo, J. D., N. M. Kumpula-McWhirter, K. J. Hotzel, M. Gonzalez and W. P. Cheevers (2004). “Glycosylation of immunodominant linear epitopes in the carboxy-terminal region of the caprine arthritis-encephalitis virus surface envelope enhances vaccine-induced type-specific and cross-reactive neutralizing antibody responses.” J Virol 78(17): 9190-9202.
(56) Truong, C., D. Mahe, P. Blanchard, M. Le Dimna, F. Madec, A. Jestin and E. Albina (2001). “Identification of an immunorelevant ORF2 epitope from porcine circovirus type 2 as a serological marker for experimental and natural infection.” Arch Virol 146(6): 1197-1211.
(57) Worsfold, C. S., R. Dardari, S. Law, M. Eschbaumer, N. Nourozieh, F. Marshall and M. Czub (2015). “Assessment of neutralizing and non-neutralizing antibody responses against Porcine circovirus 2 in vaccinated and non-vaccinated farmed pigs.” J Gen Virol 96(9): 2743-2748.
(58) Xiao, C. T., P. G. Halbur and T. Opriessnig (2012). “Complete genome sequence of a novel porcine circovirus type 2b variant present in cases of vaccine failures in the United States.” J Virol 86(22): 12469.
(59) Xiao, C. T., P. G. Halbur and T. Opriessnig (2015). “Global molecular genetic analysis of porcine circovirus type 2 (PCV2) sequences confirms the presence of four main PCV2 genotypes and reveals a rapid increase of PCV2d.” J Gen Virol 96(Pt 7): 1830-1841.
(60) Yoon, K. J., J. J. Zimmerman, S. L. Swenson, M. J. McGinley, K. A. Eernisse, A. Brevik, L. L. Rhinehart, M. L. Frey, H. T. Hill and K. B. Platt (1995). “Characterization of the humoral immune response to porcine reproductive and respiratory syndrome (PRRS) virus infection.” J Vet Diagn Invest 7(3): 305-312.
(61) Zost, S. J., N. C. Wu, S. E. Hensley and I. A. Wilson (2019). “Immunodominance and Antigenic Variation of Influenza Virus Hemagglutinin: Implications for Design of Universal Vaccine Immunogens.” J Infect Dis 219(Suppl 1): S38-S45.

1. An immunogenic composition, comprising:
a PCV2 infectious clone with a re-engineered PCV2 capsid in the backbone thereof;
wherein the re-engineered PCV2 capsid includes a modified immunogenic region.

2. The composition of claim 1, wherein the PCV2 infectious clone is selected from the group consisting of PCV2a (SEQ ID NO: 1), PCV2b (SEQ ID NO: 2), and PCV2d (SEQ ID NO: 41).

3. The composition of claim 1, wherein the modified immunogenic region includes at least one modification as compared to a region selected from the group consisting of wild type region 1, wild type region 2, wild type region 3, wild type region 4, and combinations thereof.

4. The composition of claim 1, wherein the modified immunogenic region includes at least one modification to a decoy epitope sequence contained therein.

5. The composition of claim 4, wherein the decoy epitope sequence is selected from the group consisting of SEQ ID NO: 3, SEQ ID NO: 4, SEQ ID NO: 5, SEQ ID NO: 6, SEQ ID NO: 17, SEQ ID NO: 18, SEQ ID NO: 20, SEQ ID NO: 25, SEQ ID NO: 26, and combinations thereof.

6. The composition of claim 4, wherein the decoy epitope sequence is selected from the group consisting of SEQ ID NO: 3, SEQ ID NO: 17, SEQ ID NO: 18, and combinations thereof.

7. The composition of claim 4, wherein the decoy epitope sequence is selected from the group consisting of SEQ ID NO: 4, SEQ ID NO: 5, SEQ ID NO: 20, and combinations thereof.

8. The composition of claim 7, wherein the modified immunogenic region includes at least one modification to each of SEQ ID NO: 5 and SEQ ID NO: 20.

9. The composition of claim 8, wherein the modified immunogenic region includes at least two modifications to each of SEQ ID NO: 5 and SEQ ID NO: 20.

10. The composition of claim 4, wherein the decoy epitope sequence is selected from the group consisting of SEQ ID NO: 25, SEQ ID NO: 26, and a combination thereof.

11. The composition of claim 1, wherein the modified immunogenic region includes a modified decoy epitope sequence selected from the group consisting of SEQ ID NO: 23, SEQ ID NO: 24, and a combination thereof.

12. The composition of claim 1, wherein the re-engineered PCV2 capsid further comprises at least one modified serine or modified leucine codon;
wherein the modified serine codon include at least one mutation selected from the group consisting of UCA to UAA, UCA to UGA, and UCG to UAG; and
wherein the modified leucine codon include at least one mutation selected from the group consisting of UUA to UAA, UUA to UGA, and UUG to UAG.

13. The composition of claim 12, wherein each serine and leucine codon is modified.

14. The composition of claim 12, wherein the mutation converts the at least one modified serine or modified leucine to a stop codon.

15. The composition of claim 1, further comprising a marker for differentiating infected and vaccinated animals (DIVA).

16. The composition of claim 15, wherein the DIVA marker includes a peptide that is foreign to swine.

17. The composition of claim 16, wherein the DIVA marker includes SEQ ID NO: 27.

18. A method of vaccinating against PCV2, the method comprising administering the composition according to claim 1 to a subject in need thereof.

19. The method of claim 18, wherein after administration the PCV2 infectious clone with the re-engineered PCV2 capsid in the backbone thereof refocus the immune response in the subject towards more protective regions on the capsid protein.

20. The method of claim 18, further comprising determining whether the subject is infected using the DIVA marker and removing infected subject from the herd.
